(19) United States
(12) Patent Application Publication
(10) Pub. No.: US 2012/0053211 A1
(43) Pub. Date: Mar. 1, 2012

(54) TREATMENT OF PANCREATIC CANCER

(75) Inventor: Mark S. Chapman, San Diego, CA (US)

(73) Assignee: ARDEA BIOSCIENCES, INC., San Diego, CA (US)

(21) Appl. No.: 13/255,331

(22) PCT Filed: Mar. 11, 2010
(86) PCT No.: PCT/US2010/027021

§ 371 Date: Nov. 17, 2011

Related U.S. Application Data
(60) Provisional application No. 61/159,397, filed on Mar. 11, 2009.

(51) Int. Cl. A61K 31/4412; A61P 35/04; A61P 35/00; A61K 31/18; C12N 5/09

(52) U.S. Cl. 514/347; 514/601; 435/375

(57) ABSTRACT

The present invention relates to the field of oncology and relates to use of (S)-N-(3,4-difluoro-2-(2-fluoro-4-iodophenylamino)-6-methoxyphenyl)-1-(2,3-dihydroxypropyl)cyclopropane-1-sulfonamide or a pharmaceutically acceptable salt thereof or N-(4-(2-fluoro-4-iodophenylamino)-1,5-dimethyl-6-oxo-1,6-dihydropyridin-3-yl)cyclopropanesulfonamide or a polymorph thereof, or a pharmaceutically acceptable salt thereof, or pharmaceutical compositions comprising the same, for the preparation of a medicament for the treatment of pancreatic cancer. Further, the present invention provides methods for administering a compound disclosed herein to an individual in need thereof by administering (S)-N-(3,4-difluoro-2-(2-fluoro-4-iodophenylamino)-6-methoxyphenyl)-1-(2,3-dihydroxypropyl)cyclopropane-1-sulfonamide or a pharmaceutically acceptable salt thereof or N-(4-(2-fluoro-4-iodophenylamino)-1,5-dimethyl-6-oxo-1,6-dihydropyridin-3-yl)cyclopropanesulfonamide, or a polymoph thereof, or a pharmaceutically acceptable salt thereof, or pharmaceutical compositions comprising the same.

Disclosed herein, in accordance with a first aspect of the present invention, in certain embodiments, is a method for treating a proliferative disorder of a plurality of pancreatic cells, comprising administering to an individual in need thereof a therapeutically effective amount of (S)-N-(3,4-difluoro-2-(2-fluoro-4-iodophenylamino)-6-methoxyphenyl)-1-(2,3-dihydroxypropyl)cyclopropane-1-sulfonamide; N-(4-(2-fluoro-4-iodophenylamino)-1,5-dimethyl-6-oxo-1,6-dihydropyridin-3-yl)cyclopropanesulfonamide; pharmaceutically acceptable salts of either compound; polymorphs of either compound (see e.g., U.S. patent application Ser. No. 12/399,848); or combinations thereof. In some embodiments, the proliferative disorder is a pancreatic caner. In some embodiments, the proliferative disorder is a precancerous condition of the pancreas. In some embodiments, the proliferative disorder is hyperplasia of the pancreas. In some embodiments, the proliferative disorder is metaplasia of the pancreas. In some embodiments, the proliferative disorder is dysplasia of the pancreas. In some embodiments, the proliferative disorder is duct-cell carcinoma, pleomorphic giant-cell carcinoma, giant-cell carcinoma (osteoclastoid type), cancer, adenosquamous carcinoma, mucinous (colloid) carcinoma, cystcancer, acinar-cell cancer, papillary cancer, small-cell (oat-cell) carcinoma, pancreaticoblastoma, mixed-cell carcinoma, anaplastic carcinoma, pancreatic hyperplasia, pancreatic metaplasia, pancreatic dysplasia, mucinous cystadenoma, intraductal papillary neoplasm, serous cystadenoma, papillary-cystic neoplasm, mucinous cystic tumor with dysplasia, intraductal papillary mucinous tumor with dysplasia, pseudopapillary solid tumor or a combination thereof. In some embodiments, the proliferative disorder is metastatic pancreatic cancer. In some embodiments, the administration is parenteral, by injection, intravenous, oral, topical or a combination thereof. In some embodiments, the administration is oral.

Disclosed herein, in certain embodiments, is a method of treating a pancreatic tumor, comprising administering to a subject with a pancreatic tumor a therapeutically effective amount of (S)-N-(3,4-difluoro-2-(2-fluoro-4-iodophenylamino)-6-methoxyphenyI)-1-(2,3-dihydroxypropyl)cyclopropane-1-sulfonamide; N-(4-(2-fluoro-4-iodophenylamino)-1,5-dimethyl-6-oxo-1,6-dihydropyridin-3-yl)cyclopropanesulfonamide; pharmaceutically acceptable salts of either compound; polymorphs of either compound (see e.g., U.S. patent application Ser. No. 12/399,848); or combinations thereof. In some embodiments, the tumor is benign. In some embodiments, the tumor is malignant. In some embodiments, tumor growth rate is reduced. In some embodiments, an increase in tumor size is prevented. In some embodiments, the tumor size is reduced. In some embodiments, an increase in tumor volume is prevented. In some embodiments, the tumor volume is reduced. In some embodiments, tumor proliferation is prevented. In some embodiments, tumor proliferation is reduced. In some embodiments, cell death is induced. In some embodiments, apoptosis is induced.

Disclosed herein, in certain embodiments, is a method for degrading, inhibiting the growth of, inhibiting the proliferation of or killing pancreatic cancer cells comprising contacting the cells with an amount of (S)-N-(3,4-difluoro-2-(2-fluoro-4-iodophenylamino)-6-methoxyphenyl)-1-(2,3-dihydroxypropyl)cyclopropane-1-sulfonamide; N-(4-(2-fluoro-4-iodophenylamino)-1,5-dimethyl-6-oxo-1,6-dihydropyridin-3-yl)cyclopropanesulfonamide; pharmaceutically acceptable salts of either compound; polymorphs of either compound (see e.g., U.S. patent application Ser. No. 12/399,848); or combinations thereof.

Disclosed herein, in certain embodiments, is a method for slowing the progression of pancreatic carcinogenesis, reversing pancreatic carcinogenesis or inhibiting pancreatic carcinogenesis in a subject, comprising administering to the subject an effective amount of (S)-N-(3,4-difluoro-2-(2-fluoro-4-iodophenylamino)-6-methoxyphenyl)-1-(2,3-dihydroxypropyl)cyclopropane-1-sulfonamide or a pharmaceutically acceptable salt thereof.

Disclosed herein, in certain embodiments, is a method for lowering the risk of developing invasive pancreatic cancer, comprising administering to an individual in need thereof an effective amount of (S)-N-(3,4-difluoro-2-(2-fluoro-4-iodophenylamino)-6-methoxyphenyl)-1-(2,3-dihydroxypropyl)cyclopropane-1-sulfonamide; N-(4-(2-fluoro-4-iodophenylamino)-1,5-dimethyl-6-oxo-1,6-dihydropyridin-3-yl)cyclopropanesulfonamide; pharmaceutically acceptable salts of either compound; polymorphs of either compound (see e.g., U.S. patent application Ser. No. 12/399,848); or combinations thereof. In some embodiments, the individual suffers from a disease or condition predisposing the individual to develop an invasive pancreatic cancer. In some embodiments, the individual suffers from diabetes mellitus or pancreatitis. In some embodiments, the individual suffers from a hereditary syndrome. In some embodiments, the individual suffers from hereditary nonpolyposis colorectal cancer (HNPCC) or familial adenomatous polyposis (FAP). In some embodiments, the individual has a gene mutation. In some embodiments, the individual has a gene mutation in the MSH2, MSH6, MLH1, or APC gene.

In accordance with a second aspect, the present invention relates to the use of (S)-N-(3,4-difluoro-2-(2-fluoro-4-iodophenylamino)-6-methoxyphenyl)-1-(2,3-dihydroxypropyl)cyclopropane-1-sulfonamide, or of a pharmaceutically acceptable salt thereof, or of a polymorph of (S)-N-(3,4-difluoro-2-(2-fluoro-4-iodophenylamino)-6-methoxyphenyl)-1-(2,3-dihydroxypropyl)cyclopropane-1-sulfonamide, or of a pharmaceutically acceptable salt thereof, or of N-(4-(2-fluoro-4-iodophenylamino)-1,5-dimethyl-6-oxo-1,6-dihydropyridin-3-yl)cyclopropanesulfonamide, or of a pharmaceutically acceptable salt thereof, or of a polymorph of N-(4-(2-fluoro-4-iodophenylamino)-1,5-dimethyl-6-oxo-1,6-dihydropyridin-3-yl)cyclopropanesulfonamide, or of a pharmaceutically acceptable salt thereof, for the preparation of a medicament for the treatment of a proliferative disorder of a plurality of pancreatic cells in an individual.

In accordance with an embodiment of the second aspect, the present invention relates to the above-mentioned use, wherein the proliferative disorder is a pancreatic caner.

In accordance with an embodiment of the second aspect, the present invention relates to the above-mentioned use, wherein the proliferative disorder is a precancerous condition of the pancreas.

In accordance with an embodiment of the second aspect, the present invention relates to the above-mentioned use, wherein the proliferative disorder is hyperplasia of the pancreas.

In accordance with an embodiment of the second aspect, the present invention relates to the above-mentioned use, wherein the proliferative disorder is metaplasia of the pancreas.

In accordance with an embodiment of the second aspect, the present invention relates to the above-mentioned use, wherein the proliferative disorder is dysplasia of the pancreas.

In accordance with an embodiment of the second aspect, the present invention relates to the above-mentioned use, wherein the proliferative disorder is duct-cell carcinoma, pleomorphic giant-cell carcinoma, giant-cell carcinoma (osteoclastoid type), cancer, adenosquamous carcinoma, mucinous (colloid) carcinoma, cystcancer, acinar-cell cancer, papillary cancer, small-cell (oat-cell) carcinoma, pancreaticoblastoma, mixed-cell carcinoma, anaplastic carcinoma, pancreatic hyperplasia, pancreatic metaplasia, pancreatic dysplasia, mucinous cystadenoma, intraductal papillary neoplasm, serous cystadenoma, papillary-cystic neoplasm, mucinous cystic tumor with dysplasia, intraductal papillary mucinous tumor with dysplasia, pseudopapillary solid tumor or a combination thereof.

In accordance with an embodiment of the second aspect, the present invention relates to the above-mentioned use, wherein the proliferative disorder is metastatic pancreatic cancer.

In accordance with an embodiment of the second aspect, the present invention relates to the above-mentioned use, wherein the administration is parenteral, by injection, intravenous, oral, topical or a combination thereof.

In accordance with an embodiment of the second aspect, the present invention relates to the above-mentioned use, wherein the administration is oral.

In accordance with a third aspect, the present invention relates to the use of (S)-N-(3,4-difluoro-2-(2-fluoro-4-iodophenylamino)-6-methoxyphenyl)-1-(2,3-dihydroxypropyl)cyclopropane-1-sulfonamide, or of a pharmaceutically acceptable salt thereof, or of a polymorph of (S)-N-(3,4-difluoro-2-(2-fluoro-4-iodophenylamino)-6-methoxyphenyl)-1-(2,3-dihydroxypropyl)cyclopropane-1-sulfonamide, or of a pharmaceutically acceptable salt thereof, or of N-(4-(2-fluoro-4-iodophenylamino)-1,5-dimethyl-6-oxo-1,6-dihydropyridin-3-yl)cyclopropanesulfonamide, or of a pharmaceutically acceptable salt thereof, or of a polymorph of N-(4-(2-fluoro-4-iodophenylamino)-1,5-dimethyl-6-oxo-1,6-dihydropyridin-3-yl)cyclopropanesulfonamide, or of a pharmaceutically acceptable salt thereof, for the preparation of a medicament for the treatment of a pancreatic tumor in an individual.

In accordance with an embodiment of the third aspect, the present invention relates to the above-mentioned use, wherein the tumor is benign.

In accordance with an embodiment of the third aspect, the present invention relates to the above-mentioned use, wherein the tumor is malignant.

In accordance with an embodiment of the third aspect, the present invention relates to the above-mentioned use, wherein tumor growth rate is reduced.

In accordance with an embodiment of the third aspect, the present invention relates to the above-mentioned use, wherein an increase in tumor size is prevented.

In accordance with an embodiment of the third aspect, the present invention relates to the above-mentioned use, wherein tumor size is reduced.

In accordance with an embodiment of the third aspect, the present invention relates to the above-mentioned use, wherein an increase in tumor volume is prevented.

In accordance with an embodiment of the third aspect, the present invention relates to the above-mentioned use, wherein the tumor volume is reduced.

In accordance with an embodiment of the third aspect, the present invention relates to the above-mentioned use, wherein tumor proliferation is prevented.

In accordance with an embodiment of the third aspect, the present invention relates to the above-mentioned use, wherein tumor proliferation is reduced.

In accordance with an embodiment of the third aspect, the present invention relates to the above-mentioned use, wherein cell death is induced.

In accordance with an embodiment of the third aspect, the present invention relates to the above-mentioned use, wherein apoptosis is induced.

In accordance with a fourth aspect, the present invention relates to the use of (S)-N-(3,4-difluoro-2-(2-fluoro-4-iodophenylamino)-6-methoxyphenyl)-1-(2,3-dihydroxypropyl)cyclopropane-1-sulfonamide, or of a pharmaceutically acceptable salt thereof, or of a polymorph of (S)-N-(3,4-difluoro-2-(2-fluoro-4-iodophenylamino)-6-methoxyphenyl)-1-(2,3-dihydroxypropyl)cyclopropane-1-sulfonamide, or of a pharmaceutically acceptable salt thereof, or of N-(4-(2-fluoro-4-iodophenylamino)-1,5-dimethyl-6-oxo-1,6-dihydropyridin-3-yl)cyclopropanesulfonamide, or of a pharmaceutically acceptable salt thereof, or of a polymorph of N-(4-(2-fluoro-4-iodophenylamino)-1,5-dimethyl-6-oxo-1,6-dihydropyridin-3-yl)cyclopropanesulfonamide, or of a pharmaceutically acceptable salt thereof, for the preparation of a medicament for inhibiting the proliferation of or killing pancreatic cancer cells in an individual.

In accordance with a fifth aspect, the present invention relates to the use of (S)-N-(3,4-difluoro-2-(2-fluoro-4-iodophenylamino)-6-methoxyphenyl)-1-(2,3-dihydroxypropyl)cyclopropane-1-sulfonamide, or of a pharmaceutically acceptable salt thereof, or of a polymorph of (S)-N-(3,4-difluoro-2-(2-fluoro-4-iodophenylamino)-6-methoxyphenyl)-1-(2,3-dihydroxypropyl)cyclopropane-1-sulfonamide, or of a pharmaceutically acceptable salt thereof, or of N-(4-(2-fluoro-4-iodophenylamino)-1,5-dimethyl-6-oxo-1,6-dihydropyridin-3-yl)cyclopropanesulfonamide, or of a pharmaceutically acceptable salt thereof, or of a polymorph of N-(4-(2-fluoro-4-iodophenylamino)-1,5-dimethyl-6-oxo-1,6-dihydropyridin-3-yl)cyclopropanesulfonamide, or of a pharmaceutically acceptable salt thereof, for the preparation of a medicament for slowing the progression of pancreatic carcinogenesis, reversing pancreatic carcinogenesis or inhibiting pancreatic carcinogenesis in an individual.

In accordance with a sixth aspect, the present invention relates to the use of (S)-N-(3,4-difluoro-2-(2-fluoro-4-iodophenylamino)-6-methoxyphenyl)-1-(2,3-dihydroxypropyl)cyclopropane-1-sulfonamide, or of a pharmaceutically acceptable salt thereof, or of a polymorph of (S)-N-(3,4-difluoro-2-(2-fluoro-4-iodophenylamino)-6-methoxyphenyl)-1-(2,3-dihydroxypropyl)cyclopropane-1-sulfonamide, or of a pharmaceutically acceptable salt thereof, or of N-(4-(2-fluoro-4-iodophenylamino)-1,5-dimethyl-6-oxo-1,6-dihydropyridin-3-yl)cyclopropanesulfonamide, or of a pharmaceutically acceptable salt thereof, or of a polymorph of N-(4-(2-fluoro-4-iodophenylamino)-1,5-dimethyl-6-oxo-1,6-dihydropyridin-3-yl)cyclopropanesulfonamide, or of a pharmaceutically acceptable salt thereof, for the preparation of a medicament for lowering the risk of developing invasive pancreatic cancer in an individual.

In accordance with an embodiment of the sixth aspect, the present invention relates to the above-mentioned use, wherein the individual suffers from a disease or condition predisposing the individual to develop an invasive pancreatic cancer.

In accordance with an embodiment of the sixth aspect, the present invention relates to the above-mentioned use, wherein the individual suffers from diabetes mellitus or pancreatitis.

In accordance with an embodiment of the sixth aspect, the present invention relates to the above-mentioned use, wherein the individual suffers from a hereditary syndrome.

In accordance with an embodiment of the sixth aspect, the present invention relates to the above-mentioned use, wherein the individual suffers from hereditary nonpolyposis colorectal cancer (HNPCC) or familial adenomatous polyposis (FAP).

In accordance with an embodiment of the sixth aspect, the present invention relates to the above-mentioned use, wherein the individual has a gene mutation.

In accordance with an embodiment of the sixth aspect, the present invention relates to the above-mentioned use, wherein the individual has a gene mutation in the MSH2, MSH6, MLH1, or APC gene.

Disclosed herein, in certain embodiments, is a kit for treating a proliferative disorder of a plurality of pancreatic cells in an individual in need thereof, comprising: (a) (S)-N-(3,4-difluoro-2-(2-fluoro-4-iodophenylamino)-6-methoxyphenyl)-1-(2,3-dihydroxypropyl)cyclopropane-1-sulfonamide; N-(4-(2-fluoro-4-iodophenylamino)-1,5-dimethyl-6-oxo-1,6-dihydropyridin-3-yl)cyclopropanesulfonamide;

pharmaceutically acceptable salts of either compound; polymorphs of either compound (see e.g., U.S. patent application Ser. No. 12/399,848); or combinations thereof; and (b) instructions for administration of S)-N-(3,4-difluoro-2-(2-fluoro-4-iodophenylamino)-6-methoxyphenyl)-1-(2,3-dihydroxypropyl)cyclopropane-1-sulfonamide; N-(4-(2-fluoro-4-iodophenylamino)-1,5-dimethyl-6-oxo-1,6-dihydropyridin-3-yl)cyclopropanesulfonamide; pharmaceutically acceptable salts of either compound; polymorphs of either compound (see e.g., U.S. patent application Ser. No. 12/399,848); or combinations thereof.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 presents Tumor growth curves showing the group median tumor volumes as a function of time (days).

FIG. 2 presents Body weight change curves showing the group median % body weight change as a function of time (days).

FIG. 3 presents the decrease in tumor volume following administration of compound A as a function of time.

FIG. 4 presents the decrease in tumor volume following administration of compound A as a function of time.

### DETAILED DESCRIPTION OF THE INVENTION

Certain Definitions

The term “subject”, “individual” or “individual” as used herein encompasses mammals and non-mammals. None of the terms are to be construed as requiring the supervision of a medical professional (e.g., a physician, nurse, orderly, hospice worker). Examples of mammals include, but are not limited to, any member of the Mammalian class: humans, non-human primates (e.g., chimpanzees, and other apes and monkey species); farm animals (e.g., cattle, horses, sheep, goats, swine); domestic animals (e.g., rabbits, dogs, and cats); laboratory animals including rodents, (e.g., rats, mice and guinea pigs), and the like. Examples of non-mammals include, but are not limited to, birds, fish and the like. In one embodiment of the methods and compositions provided herein, the mammal is a human.

The terms “treat,” “treating” or “treatment,” and other grammatical equivalents mean slowing or stopping the development of a disorder, causing regression of a disorder, ameliorating the symptoms of a disorder, preventing the development or presentation of additional symptoms, ameliorating and/or preventing the underlying cause of a symptom, or combinations thereof. The term further includes achieving a prophylactic benefit. For prophylactic benefit, a compound or composition disclosed herein is administered to an individual at risk of developing a particular disorder, predisposed to developing a particular disorder, or to an individual reporting one or more of the physiological symptoms of a disorder.

The terms “effective amount”, “therapeutically effective amount” or “pharmaceutically effective amount” as used herein, refer to an amount of an agent or compound that is sufficient to treat a disorder. In some embodiments, the result is a reduction in and/or alleviation of the signs, symptoms, or causes of a disorder, or any other desired alteration of a biological system. For example, an “effective amount” for therapeutic uses is the amount of the composition comprising a compound as disclosed herein required to provide a clinically significant decrease in a disorder. An appropriate “effective” amount in any individual case is determined using any suitable technique, (e.g., a dose escalation study).

The term “pharmaceutically acceptable” as used herein, refers to a material, (e.g., a carrier or diluent), which does not abrogate the biological activity or properties of the compounds described herein, and is relatively nontoxic (i.e., the material is administered to an individual without causing undesirable biological effects or interacting in a deleterious manner with any of the components of the composition in which it is contained).

As used herein, the term “proliferative disorder” refers to a disorder wherein the growth of a population of cells exceeds, and is uncoordinated with, that of the surrounding cells. In certain instances, a proliferative disorder leads to the formation of a tumor. In some embodiments, the tumor is benign, pre-malignant, or malignant. In some embodiments, the proliferative disorder is a pancreatic cancer. In some embodiments, the proliferative disorder is a pre-malignant growth on the pancreas.

As used herein, the term “selectively” means tending to occur at a higher frequency in one population than in another population.

Proliferative Disorders of Pancreatic Cells

Disclosed herein, in certain embodiments, is a method for treating a proliferative disorder. In some embodiments, the proliferative disorder is a proliferative disorder of a plurality of pancreatic cells. In some embodiments, the proliferative disorder is a tumor. In some embodiments, the proliferative disorder is benign. In some embodiments, the proliferative disorder is malignant. In some embodiments, the proliferative disorder is pancreatic cancer. In some embodiments, the proliferative disorder is pre-cancerous.

As used herein, the phrase “proliferative disorder of a plurality of pancreatic cells” includes, but is not limited to, hyperplasia, metaplasia, and dysplasia of the pancreas. The phrase also includes mucinous cystadenoma, intraductal papillary neoplasm, serous cystadenoma, papillary-cystic neoplasm, mucinous cystic tumor with dysplasia, intraductal papillary mucinous tumor with dysplasia, and pseudopapillary solid tumor.

In certain instances, diabetes mellitus or pancreatitis predisposes an individual to develop a proliferative disorder of a plurality of pancreatic cells. In certain instances, individuals are at an increased risk of developing a proliferative disorder of a plurality of pancreatic cells due to a hereditary syndrome selected from the group consisting of hereditary nonpolyposis colorectal cancer (HNPCC) and familial adenomatous polyposis (FAP). In certain instances, individuals are at an increased risk of developing a proliferative disorder of a plurality of pancreatic cells due to a mutation in a gene selected from the group consisting of MSH2, MSH6, MLH1, and APC.

The Pancreas

Located in the upper abdomen (in the retroperitoneum), the pancreas is a dual-function gland of the digestive and endocrine system. In certain instances, the pancreas functions as an endocrine gland (e.g., producing several important hormones). In certain instances, the pancreas functions as an exocrine gland (e.g., secreting fluids containing digestive enzymes that pass to the small intestine).

Pancreatic Cancer Pancreatic cancer is the fourth most common cause of cancer death in the US (after lung, colon and breast), comprising 6% of all cancer-related deaths. In 2008, an estimated 37,680 new cases of pancreatic cancer will have been diagnosed in the US, with 34,290 deaths. Incidence of the disease, rises linearly after age 50, with the only definitive risk factor being cigarette smoking (smokers are four times more likely to develop the disease than non-smokers). Invasive pancreatic cancer is almost always fatal. The collective median survival time of all patients is 4-6 months. Relative 1-year survival is 24%; the overall 5-year survival rate <5%.

Pancreatic cancer is asymptomatic in its early stage and often remains undiagnosed for several months (less than one third of patients being diagnosed within 2 months of the onset symptoms). In certain instances, the delayed diagnosis results in (either partially or fully) metastasis of the cancerous cells to the liver or lymph nodes.

Currently, surgery (resectioning of the pancreas) is the primary and only curative therapy for pancreatic cancer. However, only 15-25% of tumors are resectable at the time of diagnosis and only 10-20% of patients undergoing surgery survive more than two years. Once tumor infiltration occurs and other tissues have been affected, surgery is no longer possible.

Ideally, effective treatment of pancreatic cancer should (i) control the primary tumor mass, both initially and subsequently, and (ii) treat the metastatic tumor cells. Chemoprevention (the administration of agents such as drugs, biologics, nutrients and the like) slows the progression of, reverses, or inhibits carcinogenesis, thereby lowering the risk of developing invasive or clinically significant disease.

Disclosed herein, in certain embodiments, is a method of treating pancreatic cancer. As used herein, “pancreatic cancer” includes forms of cancer of the pancreas. In some embodiments, the pancreatic cancer is metastatic pancreatic cancer. In some embodiments, the pancreatic cancer is a carcinoma, sarcoma, cancer, or combinations thereof. In some embodiments, a pancreatic cancer to be treated includes sporadic and hereditary pancreatic cancers. In some embodiments, the pancreatic cancer is duct cell carcinoma, acinar cell carcinoma, papillary mucinous carcinoma, signet ring carcinoma, adenosquamous carcinoma, undifferentiated carcinoma, mucinous carcinoma, giant cell carcinoma, small cell carcinoma, cystcancer, serous cystcancer, mucinous cystcancer, unclassified pancreatic cancer, pancreatoblastoma, or combinations thereof.

In some embodiments, an individual in need of treatment for pancreatic cancer is equal to or older than 30 years old. In some embodiments, an individual in need of treatment for pancreatic cancer is younger than 30 years old. In some embodiments, an individual in need of treatment for pancreatic cancer is equal to or older than 50 years old. In some embodiments, an individual in need of treatment for pancreatic cancer is younger than 50 years old. In some embodiments, an individual in need of treatment for pancreatic cancer is equal to or older than 70 years old. In some embodiments, an individual in need of treatment for pancreatic cancer is younger than 70 years old.

In some embodiments, an individual in need of treatment for pancreatic cancer presents with a localized tumor of the pancreas. In some embodiments, an individual in need of treatment for pancreatic cancer presents with a negative regional lymph node biopsy. In some embodiments, an individual in need of treatment for pancreatic cancer presents with a positive regional lymph node biopsy. In some embodiments, an individual in need of treatment for pancreatic cancer presents with a nodal negative pancreatic tumor (e.g., node-negative). In some embodiments, an individual in need of treatment for pancreatic cancer presents with a nodal positive tumor (e.g., node-positive).

In some embodiments, the pancreatic cancer in an individual in need of treatment for pancreatic cancer has metastasized to other locations in the body. In some embodiments, the pancreatic cancer has metastasized to a location selected from the group consisting of lymph node, stomach, bile duct, liver, bone, ovary, peritoneum and brain.

In some embodiments, any suitable method is used to identify and/or classify a pancreatic tumor, cancerous pancreatic cells, or precancerous pancreatic cells.

In some embodiments, cancer cells or precancerous cells are identified by histological typing or grading of a tissue sample (e.g., a biopsy sample). In some embodiments, cancer cells or precancerous cells are identified through the use of appropriate molecular markers.

In some embodiments, the pancreatic cancer in an individual in need of treatment for pancreatic cancer is classified according to a characteristic selected from the group consisting of: metastatic, limited stage, extensive stage, unresectable, resectable, locally advanced, localized, regional, local-regional, locally advanced, distant, multicentric, bilateral, ipsilateral, contralateral, newly diagnosed, recurrent, and inoperable.

In some embodiments, the pancreatic cancer in an individual in need of treatment for pancreatic cancer is staged according to the American Joint Committee on Cancer (AJCC) TNM classification system, where the tumor (T) has been assigned a stage of Tx, T1, T2, T3, T4; and where the regional lymph nodes (N) have been assigned a stage of NX, NO, N1; and where distant metastasis (M) has been assigned a stage of MX, MO, or M1. In some embodiments, the pancreatic cancer in an individual in need of treatment for pancreatic cancer is staged as Stage 0, I, IA, IB, II, IIA, IIB, Ill, and IV pancreatic cancer. In some embodiments, the pancreatic cancer in an individual in need of treatment for pancreatic cancer is staged as Grade GX (e.g., grade cannot be assessed), Grade 1, Grade 2, Grade 3 or Grade 4.

In some embodiments, pancreatic cancer includes a tumor that is less than or equal to about 2 centimeters in diameter. In some embodiments, pancreatic cancer includes a tumor that is from about 2 to about 5 centimeters in diameter. In some embodiments, pancreatic cancer includes a tumor that is greater than or equal to about 2 centimeters in diameter. In some embodiments, pancreatic cancer includes a tumor that is greater than 5 centimeters in diameter.

In some embodiments, pancreatic cancer is classified by microscopic appearance. In some embodiments, pancreatic cancer is classified as: well differentiated, moderately differentiated, poorly differentiated, or undifferentiated. In some embodiments, pancreatic cancer is classified by microscopic appearance with respect to mitosis count (e.g., amount of cell division) or nuclear pleiomorphism (e.g., change in cells). In some embodiments, pancreatic cancer is classified by microscopic appearance as being associated with areas of necrosis (e.g., areas of dying or degenerating cells).

In some embodiments, pancreatic cancer cell is classified as having an abnormal karyotype, having an abnormal number of chromosomes, or having one or more chromosomes that are abnormal in appearance. In some embodiments, a pancreatic cancer cell is classified as being aneuploid, triploid, tetraploid, or as having an altered ploidy. In some embodiments, a pancreatic cancer cell is classified as having a chromosomal translocation, or a deletion or duplication of an entire chromosome, or a region of deletion, duplication or amplification of a portion of a chromosome.

In some embodiments, a pancreatic cancer that is to be treated is evaluated by DNA cytometry, flow cytometry, or image cytometry. In some embodiments, a pancreatic cancer that is to be treated has been typed as having 10%, 20%, 30%, 40%, 50%, 60%, 70%, 80%, or 90% of cells in the synthesis stage of cell division (e.g., in S phase of cell division). In some embodiments, a pancreatic cancer that is to be treated has been typed as having a low S-phase fraction or a high S-phase fraction.

Predisposition to Developing Pancreatic Cancer

In some embodiments, an individual in need of treatment for pancreatic cancer has been typed to identify a familial or spontaneous mutation in p53, Rb, myc or ras. In some embodiments, an individual in need of treatment for pancreatic cancer has a mutation in a gene selected from the group consisting of K-Ras, p53, BRCA2, p16 (CDKN2A), MADH4 (DPC4), STK11, MSH2, MSH6, MLH1, and APC.

In some embodiments, an individual in need of treatment for pancreatic cancer presents with elevated levels of expression of a growth factor selected from the group consisting of EGF, TGF alpha, TGF beta 1-3, aFGF, and bTGF. In some embodiments, an individual in need of treatment for pancreatic cancer presents with elevated blood levels of CEA (carcinoembryonic antigen In some embodiments, an individual in need of treatment for pancreatic cancer presents with elevated blood levels of, or increased cellular expression of, tumor marker carbohydrate antigen 19-9 (CA 19-9).

MEK

In certain instances, a proliferative disorder of a pluarality of pancreatic cells is partially or fully caused by oncogenic Ras signaling and its effect on cyclin kinase inhibitors such as p27^{kp1}.

is In certain instances, Ras is a signal transduction protein. In certain instances, Ras is activated by the binding of guanosine nucleotides, GTP (Guanosine triphosphate) or GDP (Guanosine diphosphate).

In certain instances, the activation of Ras results in the activation of a cascade of serine/threonine kinases. In certain instances, activated Ras activates Raf proteins. In certain instances, activated Raf proteins activate “MEKI” and “MEK2.”

MEKI and MEK2 are dual-function serine/threonine and tyrosine protein kinases that, in certain instances, activate MAPK. In certain instances, activation of MAP kinase by mitogens appears induces cellular proliferation. In certain instances, constitutive activation of MAPK induces cellular transformation. In certain instances, blockade of downstream Ras signaling, as by use of a dominant negative Raf-1 protein, inhibits mitogenesis, whether induced from cell surface receptors or from oncogenic Ras mutants.

In certain instances, inhibition of the Raf-MEK-ERK signaling pathway, elicits pancreatic cancer cell cycle arrest through induced expression of p27.

Methods of Use

Disclosed herein, in certain embodiments, is a method of treating a proliferative disorder comprising administering (S)-N-(3,4-difluoro-2-(2-fluoro-4-iodophenylamino)-6-methoxyphenyl)-1-(2,3-dihydroxypropyl)cyclopropane-1-sulfonamide; N-(4-(2-fluoro-4-iodophenylamino)-1,5-dimethyl-6-oxo-1,6-dihydropyridin-3-yl)cyclopropanesulfonamide; pharmaceutically acceptable salts of either compound; polymorphs of either compound (see e.g., U.S. patent application Ser. No. 12/399,848); or combinations thereof to an individual in need thereof. In some embodiments, the proliferative disorder is a pancreatic cancer. In some embodiments, the pancreatic cancer is metastatic pancreatic cancer. In some embodiments, the proliferative disorder is a pre-malignant growth on a pancreas.

In some embodiments, an effective amount of (S)-N-(3,4-difluoro-2-(2-fluoro-4-iodophenylamino)-6-methoxyphenyl)-1-(2,3-dihydroxypropyl)cyclopropane-1-sulfonamide or a pharmaceutically acceptable salt thereof or N-(4-(2-fluoro-4-iodophenylamino)-1,5-dimethyl-6-oxo-1,6-dihydropyridin-3-yl)cyclopropanesulfonamide or a pharmaceutically acceptable salt thereof is not significantly cytotoxic to normal cells. A therapeutically effective amount is not significantly cytotoxic to normal cells if administration of the therapeutically effective amount does not induce apoptosis in greater than 10% of normal cells. A therapeutically effective amount does not significantly affect the viability of normal cells if administration at a therapeutically effective amount does not induce cell death in greater than 10% of normal cells.

In some embodiments, administering a compound disclosed herein to an individual in need thereof, induces or activates cell death selectively in pancreatic cancer cells. In some embodiments, administration to an individual in need thereof induces or activates cell death selectively in pancreatic cancer cells. In some embodiments, contacting a cell with a compound described herein induces cell death selectively in one or more cells affected by a cell proliferative disorder of the pancreas. In some embodiments, administration induces cell death selectively in one or more cells affected by a cell proliferative disorder of the pancreas.

In some embodiments, a compound described herein, modulates the activity of a molecular target. In some embodiments, modulating refers to stimulating or inhibiting the activity of a molecular target. In some embodiments, a compound of the present invention modulates the activity of a molecular target if it stimulates or inhibits the activity of the molecular target by at least 10% relative to the activity of the molecular target under the same conditions but lacking only the presence of said compound. In some embodiments, a compound described herein modulates the activity of a molecular target if it stimulates or inhibits the activity of the molecular target by at least 25%, at least 50%, at least 2-fold, at least 5-fold, at least 10-fold, at least 20-fold, at least 50-fold, at least 100-fold relative to the activity of the molecular target under the same conditions but lacking only the presence of said compound. In some embodiments, the activity of a molecular target is measured by any reproducible means. In some embodiments, the activity of a molecular target is measured in vitro or in vivo. For example, the activity of a molecular target is measured in vitro by an enzymatic activity assay or a DNA binding assay, or the activity of a molecular target is measured in vivo by assaying for expression of a reporter gene.

In some embodiments, a compound described herein, does not significantly modulate the activity of a molecular target if the addition of the compound stimulates or inhibits the activity of the molecular target by less than 10% relative to the activity of the molecular target under the same conditions but lacking only the presence of a compound disclosed herein.

In some embodiments, administering a compound disclosed herein to an individual in need thereof results in cell death. In some embodiments, cell death results from apoptosis. In some embodiments, cell death results in a decrease of at least 10% in number of cells in a population. In some embodiments, cell death means a decrease of at least 20%; in some embodiments, a decrease of at least 30%; in some embodiments, a decrease of at least 40%; in some embodiments, a decrease of at least 50%; in some embodiments, a decrease of at least 75%.

In some embodiments, the number of cells in a population is measured by any reproducible means. In some embodiments, the number of cells in a population is measured by fluorescence activated cell sorting (FACS). In some embodiments, the number of cells in a population is measured by immunofluorescence microscopy. In some embodiments, the number of cells in a population is measured by light microscopy.

In some embodiments, the compared populations are cell populations. In some embodiments, (S)-N-(3,4-difluoro-2-(2-fluoro-4-iodophenylamino)-6-methoxyphenyl)-1-(2,3-dihydroxypropyl)cyclopropane-1-sulfonamide or a pharmaceutically acceptable salt thereof or N-(4-(2-fluoro-4-iodophenylamino)-1,5-dimethyl-6-oxo-1,6-dihydropyridin-3-yl)cyclopropanesulfonamide or a pharmaceutically acceptable salt thereof acts selectively on a cancer or precancerous cell but not on a normal cell. In some embodiments, a compound described herein, acts selectively to modulate one molecular target but does not significantly modulate another molecular target. In some embodiments, the invention provides a method for selectively inhibiting the activity of an enzyme, such as a kinase. In some embodiments, an event occurs selectively in population A relative to population B if it occurs greater than two times more frequently in population A as compared to population B. In some embodiments, an event occurs selectively if it occurs greater than five times more frequently in population A. In some embodiments, an event occurs selectively if it occurs greater than ten times more frequently in population A; in some embodiments, greater than fifty times; in some embodiments, greater than 100 times; and in some embodiments, greater than 1000 times more frequently in population A as compared to population B. For example, cell death would be said to occur selectively in cancer cells if it occurred greater than twice as frequently in cancer cells as compared to normal cells.

In some embodiments, administering a compound disclosed herein to an individual in need thereof results in a reduction in size of a tumor (i.e., “tumor regression”). In some embodiments, after treatment, tumor size is reduced by 5% or greater relative to its size prior to treatment; in some embodiments, tumor size is reduced by 10% or greater; in some embodiments, reduced by 20% or greater; in some embodiments, reduced by 30% or greater; in some embodiments, reduced by 40% or greater; in some embodiments, reduced by 50% or greater; and in some embodiments, reduced by greater than 75% or greater. In some embodiments, size of a tumor is measured by any reproducible means of measurement. In some embodiments, size of a tumor is measured as a diameter of the tumor.

In some embodiments, administering a compound disclosed herein to an individual in need thereof results in a reduction in tumor volume. In some embodiments, after treatment, tumor volume is reduced by 5% or greater relative to its size prior to treatment; in some embodiments, tumor volume is reduced by 10% or greater; in some embodiments, reduced by 20% or greater; in some embodiments, reduced by 30% or greater; in some embodiments, reduced by 40% or greater; in some embodiments, reduced by 50% or greater; and in some embodiments, reduced by greater than 75% or greater. In some embodiments, tumor volume is measured in any suitable manner.

In some embodiments, administering a compound disclosed herein to an individual in need thereof results in a decrease in the number of tumors. In some embodiments, after treatment, tumor number is reduced by 5% or greater relative to number prior to treatment; in some embodiments, tumor number is reduced by 10% or greater; in some embodiments, reduced by 20% or greater; in some embodiments, reduced by 30% or greater; in some embodiments, reduced by 40% or greater; in some embodiments, reduced by 50% or greater; and in some embodiments, reduced by greater than 75%. Number of tumors is measured by any reproducible means of measurement. In some embodiments, number of tumors is measured by counting tumors visible to the naked eye or at a specified magnification. In some embodiments, the specified magnification is 2×, 3×, 4×, 5×, 10× or 50×.

In some embodiments, administering a compound disclosed herein to an individual in need thereof results in a decrease in number of metastatic lesions in other tissues or organs distant from the primary tumor site. In some embodiments, after treatment, the number of metastatic lesions is reduced by 5% or greater relative to number prior to treatment; in some embodiments, the number of metastatic lesions is reduced by 10% or greater; in some embodiments, reduced by 20% or greater; in some embodiments, reduced by 30% or greater; in some embodiments, reduced by 40% or greater; in some embodiments, reduced by 50% or greater; and in some embodiments, reduced by greater than 75%. The number of metastatic lesions is measured by any reproducible means of measurement. In some embodiments, the number of metastatic lesions is measured by counting metastatic lesions visible to the naked eye or at a specified magnification. In some embodiments, the specified magnification is 2×, 3×, 4×, 5×, 10× or 50×.

In some embodiments, administering a compound disclosed herein to an individual in need thereof results in an increase in average survival time of a population of treated subjects in comparison to a population receiving carrier alone. In some embodiments, the average survival time is increased by more than 30 days; in some embodiments, by more than 60 days; in some embodiments, by more than 90 days; and in some embodiments, by more than 120 days. An increase in average survival time of a population is measured by any reproducible means. In some embodiments, an increase in average survival time of a population is measured, for example, by calculating for a population the average length of survival following initiation of treatment. In an another aspect, an increase in average survival time of a population is measured, for example, by calculating for a population the average length of survival following completion of a first round of treatment.

In some embodiments, administering a compound disclosed herein to an individual in need thereof results in an increase in average survival time of a population of treated subjects in comparison to a population of untreated subjects. In some embodiments, the average survival time is increased by more than 30 days; in some embodiments, by more than 60 days; in some embodiments, by more than 90 days; and in some embodiments, by more than 120 days. An increase in average survival time of a population is measured by any reproducible means. In some embodiments, an increase in average survival time of a population is measured, for example, by calculating for a population the average length of survival following initiation of treatment with an active compound. In an another aspect, an increase in average survival time of a population is measured, for example, by calculating for a population the average length of survival following completion of a first round of treatment with an active compound.

In some embodiments, administering a compound disclosed herein to an individual in need thereof results in a decrease in the mortality rate of a population of treated subjects in comparison to a population receiving carrier alone. In some embodiments, administering a compound disclosed herein to an individual in need thereof results in a decrease in the mortality rate of a population of treated subjects in comparison to an untreated population. In some embodiments, administering a compound disclosed herein to an individual in need thereof results a decrease in the mortality rate of a population of treated subjects in comparison to a population receiving monotherapy with a drug that is not (S)-N-(3,4-difluoro-2-(2-fluoro-4-iodophenylamino)-6-methoxyphenyl)-1-(2,3-dihydroxypropyl)cyclopropane-1-sulfonamide or a pharmaceutically acceptable salt thereof or N-(4-(2-fluoro-4-iodophenylamino)-1,5-dimethyl-6-oxo-1,6-dihydropyridin-3-yl)cyclopropanesulfonamide or a pharmaceutically acceptable salt thereof. In some embodiments, the mortality rate is decreased by more than 2%; in some embodiments, by more than 5%; in some embodiments, by more than 10%; and in some embodiments, by more than 25%. In some embodiments, a decrease in the mortality rate of a population of treated subjects is measured by any reproducible means. In some embodiments, a decrease in the mortality rate of a population is measured by calculating for a population the average number of disease-related deaths per unit time following initiation of treatment with an active compound. In yet another aspect, a decrease in the mortality rate of a population is measured by calculating for a population the average number of disease-related deaths per unit time following completion of a first round of treatment with an active compound.

In some embodiments, administering a compound disclosed herein to an individual in need thereof results in a decrease in tumor growth rate. In some embodiments, after treatment, tumor growth rate is reduced by at least 5% relative to number prior to treatment; in some embodiments, tumor growth rate is reduced by at least 10%; in some embodiments, reduced by at least 20%; in some embodiments, reduced by at least 30%; in some embodiments, reduced by at least 40%; in some embodiments, reduced by at least 50%; in some embodiments, reduced by at least 50%; and in some embodiments, reduced by at least 75%. Tumor growth rate is measured by any reproducible means of measurement. In some embodiments, tumor growth rate is measured according to a change in tumor diameter per unit time.

In some embodiments, administering a compound disclosed herein to an individual in need thereof results in a decrease in tumor regrowth. In some embodiments, after treatment, tumor regrowth is less than 5%; in some embodiments, tumor regrowth is less than 10%; in some embodiments, less than 20%; in some embodiments, less than 30%; in some embodiments, less than 40%; in some embodiments, less than 50%; in some embodiments, less than 50%; and in some embodiments, less than 75%. Tumor regrowth is measured by any reproducible means of measurement. In some embodiments, tumor regrowth is measured, for example, by measuring an increase in the diameter of a tumor after a prior tumor shrinkage that followed treatment. In some embodiments, a decrease in tumor regrowth is indicated by failure of tumors to reoccur after treatment has stopped.

In some embodiments, administering a compound disclosed herein to an individual in need thereof administering a compound disclosed herein to an individual in need thereof results in a reduction in the rate of cellular proliferation. In some embodiments, after treatment, the rate of cellular proliferation is reduced by at least 5%; in some embodiments, by at least 10%; in some embodiments, by at least 20%; in some embodiments, by at least 30%; in some embodiments, by at least 40%; in some embodiments, by at least 50%; in some embodiments, by at least 50%; and in some embodiments, by at least 75%. The rate of cellular proliferation is measured by any reproducible means of measurement.

In some embodiments, the rate of cellular proliferation is measured by measuring the number of dividing cells in a tissue sample per unit time.

In some embodiments, administering a compound disclosed herein to an individual in need thereof results in a reduction in the proportion of proliferating cells. In some embodiments, after treatment, the proportion of proliferating cells is reduced by at least 5%; in some embodiments, by at least 10%; in some embodiments, by at least 20%; in some embodiments, by at least 30%; in some embodiments, by at least 40%; in some embodiments, by at least 50%; in some embodiments, by at least 50%; and in some embodiments, by at least 75%. The proportion of proliferating cells is measured by any reproducible means of measurement. In some embodiments, the proportion of proliferating cells is measured by quantifying the number of dividing cells relative to the number of nondividing cells in a tissue sample. In some embodiments, the proportion of proliferating cells is equivalent to the mitotic index.

In some embodiments, administering a compound disclosed herein to an individual in need thereof results in a decrease in size of an area or zone of cellular proliferation. In some embodiments, after treatment, size of an area or zone of cellular proliferation is reduced by at least 5% relative to its size prior to treatment; in some embodiments, reduced by at least 10%; in some embodiments, reduced by at least 20%; in some embodiments, reduced by at least 30%; in some embodiments, reduced by at least 40%; in some embodiments, reduced by at least 50%; in some embodiments, reduced by at least 50%; and in some embodiments, reduced by at least 75%. Size of an area or zone of cellular proliferation is measured by any reproducible means of measurement. In some embodiments, size of an area or zone of cellular proliferation is measured as a diameter or width of an area or zone of cellular proliferation.

In some embodiments, administering a compound disclosed herein to an individual in need thereof results in a decrease in the number or proportion of cells having an abnormal appearance or morphology. In some embodiments, after treatment, the number of cells having an abnormal morphology is reduced by at least 5% relative to its size prior to treatment; in some embodiments, reduced by at least 10%; in some embodiments, reduced by at least 20%; in some embodiments, reduced by at least 30%; in some embodiments, reduced by at least 40%; in some embodiments, reduced by at least 50%; in some embodiments, reduced by at least 50%; and in some embodiments, reduced by at least 75%. An abnormal cellular appearance or morphology is measured by any reproducible means of measurement. In some embodiments, an abnormal cellular morphology is measured by microscopy, e.g., using an inverted tissue culture microscope. In some embodiments, an abnormal cellular morphology takes the form of nuclear pleiomorphism.

In some embodiments, administering a compound disclosed herein to an individual in need thereof results in one or more of the following: accumulation of cells in G1 and/or S phase of the cell cycle, cytotoxicity via cell death in cancer cells but not in normal cells, antitumor activity in animals with a therapeutic index of at least 2. As used herein, “therapeutic index” is the maximum tolerated dose divided by the efficacious dose.

In some embodiments, a compound and/or composition disclosed herein is administered to degrade, inhibit the growth of or to kill a cell. In some embodiments, the cell is a cancer cell. In some embodiments, the cell is a brain, breast, lung, ovarian, pancreatic, prostate, renal, or colorectal cancer cell.

In some embodiments, a compound and/or composition disclosed herein is administered to inhibit the growth of a target cell. In some embodiments, the growth of a target cell is about 1% inhibited relative to the growth rate preceding administration of a compound and/or composition disclosed herein. In some embodiments, the growth of a target cell is about 2% inhibited relative to the growth rate preceding administration of a compound and/or composition disclosed herein. In some embodiments, the growth of a target cell is about 3% inhibited relative to the growth rate preceding administration of a compound and/or composition disclosed herein. In some embodiments, the growth of a target cell is about 4% inhibited relative to the growth rate preceding administration of a compound and/or composition disclosed herein. In some embodiments, the growth of a target cell is about 5% inhibited relative to the growth rate preceding administration of a compound and/or composition disclosed herein. In some embodiments, the growth of a target cell is about 10% inhibited relative to the growth rate preceding administration of a compound and/or composition disclosed herein. In some embodiments, the growth of a target cell is about 20% inhibited relative to the growth rate preceding administration of a compound and/or composition disclosed herein. In some embodiments, the growth of a target cell is about 25% inhibited relative to the growth rate preceding administration of a compound and/or composition disclosed herein. In some embodiments, the growth of a target cell is about 30% inhibited relative to the growth rate preceding administration of a compound and/or composition disclosed herein. In some embodiments, the growth of a target cell is about 40% inhibited relative to the growth rate preceding administration of a compound and/or composition disclosed herein. In some embodiments, the growth of a target cell is about 50% inhibited relative to the growth rate preceding administration of a compound and/or composition disclosed herein. In some embodiments, the growth of a target cell is about 60% inhibited relative to the growth rate preceding administration of a compound and/or composition disclosed herein. In some embodiments, the growth of a target cell is about 70% inhibited relative to the growth rate preceding administration of a compound and/or composition disclosed herein. In some embodiments, the growth of a target cell is about 75% inhibited relative to the growth rate preceding administration of a compound and/or composition disclosed herein. In some embodiments, the growth of a target cell is about 80% inhibited relative to the growth rate preceding administration of a compound and/or composition disclosed herein. In some embodiments, the growth of a target cell is about 90% inhibited relative to the growth rate preceding administration of a compound and/or composition disclosed herein. In some embodiments, the growth of a target cell is about 100% inhibited relative to the growth rate preceding administration of a compound and/or composition disclosed herein. In some embodiments, the target cell is a abnormally proliferative (i.e., neoplastic) pancreatic cell.

In some embodiments, a compound and/or composition disclosed herein is administered to degrade a target cell. In some embodiments, a compound and/or composition disclosed herein is administered to degrade a plurality of target cells. In some embodiments, 1% of the target cells are degraded. In some embodiments, 2% of the target cells are degraded. In some embodiments, 3% of the target cells are degraded. In some embodiments, 4% of the target cells are degraded. In some embodiments, 5% of the target cells are degraded. In some embodiments, 10% of the target cells are degraded. In some embodiments, 20% of the target cells are degraded. In some embodiments, 25% of the target cells are degraded. In some embodiments, 30% of the target cells are degraded. In some embodiments, 40% of the target cells are degraded. In some embodiments, 50% of the target cells are degraded. In some embodiments, 60% of the target cells are degraded. In some embodiments, 70% of the target cells are degraded. In some embodiments, 75% of the target cells are degraded. In some embodiments, 80% of the target cells are degraded. In some embodiments, 90% of the target cells are degraded. In some embodiments, 100% of the target cells are degraded. In some embodiments, essentially all of the target cells are degraded. In some embodiments, the target cell is a abnormally proliferative (i.e., neoplastic) pancreatic cell.

In some embodiments, a compound and/or composition disclosed herein is administered to kill a target cell. In some embodiments, a compound and/or composition disclosed herein is administered to kill a plurality of target cells. In some embodiments, 1% of the target cells are killed. In some embodiments, 2% of the target cells are killed. In some embodiments, 3% of the target cells are killed. In some embodiments, 4% of the target cells are killed. In some embodiments, 5% of the target cells are killed. In some embodiments, 10% of the target cells are killed. In some embodiments, 20% of the target cells are killed. In some embodiments, 25% of the target cells are killed. In some embodiments, 30% of the target cells are killed. In some embodiments, 40% of the target cells are killed. In some embodiments, 50% of the target cells are killed. In some embodiments, 60% of the target cells are killed. In some embodiments, 70% of the target cells are killed. In some embodiments, 75% of the target cells are killed. In some embodiments, 80% of the target cells are killed. In some embodiments, 90% of the target cells are killed. In some embodiments, 100% of the target cells are killed. In some embodiments, the target cell is an abnormally proliferative (i.e., neoplastic) pancreatic cell.

In some embodiments, a compound and/or composition disclosed herein is administered to reduce the size of a tumor, inhibit tumor growth, reduce metastasis or prevent metastasis in an individual in need thereof.

In some embodiments, the size of a tumor is reduced. In some embodiments, the size of a tumor is reduced by at least 1%. In some embodiments, the size of a tumor is reduced by at least 2%. In some embodiments, the size of a tumor is reduced by at least 3%. In some embodiments, the size of a tumor is reduced by at least 4%. In some embodiments, the size of a tumor is reduced by at least 5%. In some embodiments, the size of a tumor is reduced by at least 10%. In some embodiments, the size of a tumor is reduced by at least 20%. In some embodiments, the size of a tumor is reduced by at least 25%. In some embodiments, the size of a tumor is reduced by at least 30%. In some embodiments, the size of a tumor is reduced by at least 40%. In some embodiments, the size of a tumor is reduced by at least 50%. In some embodiments, the size of a tumor is reduced by at least 60%. In some embodiments, the size of a tumor is reduced by at least 70%. In some embodiments, the size of a tumor is reduced by at least 75%. In some embodiments, the size of a tumor is reduced by at least 80%. In some embodiments, the size of a tumor is reduced by at least 85%. In some embodiments, the size of a tumor is reduced by at least 90%. In some embodiments, the size of a tumor is reduced by at least 95%.

In some embodiments, tumor growth is inhibited. In some embodiments, tumor growth is inhibited by at least 1% relative to the growth rate preceding administration of a compound and/or composition disclosed herein. In some embodiments, tumor growth is inhibited by at least 2% relative to the growth rate preceding administration of a compound and/or composition disclosed herein. In some embodiments, tumor growth is inhibited by at least 3% relative to the growth rate preceding administration of a compound and/or composition disclosed herein. In some embodiments, tumor growth is inhibited by at least 4% relative to the growth rate preceding administration of a compound and/or composition disclosed herein. In some embodiments, tumor growth is inhibited by at least 5% relative to the growth rate preceding administration of a compound and/or composition disclosed herein. In some embodiments, tumor growth is inhibited by at least 6% relative to the growth rate preceding administration of a compound and/or composition disclosed herein. In some embodiments, tumor growth is inhibited by at least 10% relative to the growth rate preceding administration of a compound and/or composition disclosed herein. In some embodiments, tumor growth is inhibited by at least 20% relative to the growth rate preceding administration of a compound and/or composition disclosed herein. In some embodiments, tumor growth is inhibited by at least 30% relative to the growth rate preceding administration of a compound and/or composition disclosed herein. In some embodiments, tumor growth is inhibited by at least 40% relative to the growth rate preceding administration of a compound and/or composition disclosed herein. In some embodiments, tumor growth is inhibited by at least 50% relative to the growth rate preceding administration of a compound and/or composition disclosed herein. In some embodiments, tumor growth is inhibited by at least 60% relative to the growth rate preceding administration of a compound and/or composition disclosed herein. In some embodiments, tumor growth is inhibited by at least 70% relative to the growth rate preceding administration of a compound and/or composition disclosed herein. In some embodiments, tumor growth is inhibited by at least 75% relative to the growth rate preceding administration of a compound and/or composition disclosed herein. In some embodiments, tumor growth is inhibited by at least 80% relative to the growth rate preceding administration of a compound and/or composition disclosed herein. In some embodiments, tumor growth is inhibited by at least 90% relative to the growth rate preceding administration of a compound and/or composition disclosed herein. In some embodiments, tumor growth is inhibited by at least 95% relative to the growth rate preceding administration of a compound and/or composition disclosed herein. In some embodiments, tumor growth is inhibited by at least 99% relative to the growth rate preceding administration of a compound and/or composition disclosed herein.

In some embodiments, metastasis is inhibited. In some embodiments, metastasis is inhibited by at least 1% relative to the growth rate preceding administration of a compound and/or composition disclosed herein. In some embodiments, metastasis is inhibited by at least 2% relative to the growth rate preceding administration of a compound and/or composition disclosed herein. In some embodiments, metastasis is inhibited by at least 3% relative to the growth rate preceding administration of a compound and/or composition disclosed herein. In some embodiments, metastasis is inhibited by at least 4% relative to the growth rate preceding administration of a compound and/or composition disclosed herein. In some embodiments, metastasis is inhibited by at least 5% relative to the growth rate preceding administration of a compound and/or composition disclosed herein. In some embodiments, metastasis is inhibited by at least 6% relative to the growth rate preceding administration of a compound and/or composition disclosed herein. In some embodiments, metastasis is inhibited by at least 10% relative to the growth rate preceding administration of a compound and/or composition disclosed herein. In some embodiments, metastasis is inhibited by at least 20% relative to the growth rate preceding administration of a compound and/or composition disclosed herein. In some embodiments, metastasis is inhibited by at least 30% relative to the growth rate preceding administration of a compound and/or composition disclosed herein. In some embodiments, metastasis is inhibited by at least 40% relative to the growth rate preceding administration of a compound and/or composition disclosed herein. In some embodiments, metastasis is inhibited by at least 50% relative to the growth rate preceding administration of a compound and/or composition disclosed herein. In some embodiments, metastasis is inhibited by at least 60% relative to the growth rate preceding administration of a compound and/or composition disclosed herein. In some embodiments, metastasis is inhibited by at least 70% relative to the growth rate preceding administration of a compound and/or composition disclosed herein. In some embodiments, metastasis is inhibited by at least 75% relative to the growth rate preceding administration of a compound and/or composition disclosed herein. In some embodiments, metastasis is inhibited by at least 80% relative to the growth rate preceding administration of a compound and/or composition disclosed herein. In some embodiments, metastasis is inhibited by at least 90% relative to the growth rate preceding administration of a compound and/or composition disclosed herein. In some embodiments, metastasis is inhibited by at least 95% relative to the growth rate preceding administration of a compound and/or composition disclosed herein. In some embodiments, metastasis is inhibited by at least 99% relative to the growth rate preceding administration of a compound and/or composition disclosed herein. In some embodiments, metastasis is prevented.

Pharmaceutical Compositions

Disclosed herein, in certain embodiments, is a pharmaceutical composition comprising (S)-N-(3,4-difluoro-2-(2-fluoro-4-iodophenylamino)-6-methoxyphenyl)-1-(2,3-dihydroxypropyl)cyclopropane-1-sulfonamide; N-(4-(2-fluoro-4-iodophenylamino)-1,5-dimethyl-6-oxo-1,6-dihydropyridin-3-yl)cyclopropanesulfonamide; pharmaceutical salts thereof; or combinations thereof. In some embodiments, the composition is administered to treat a proliferative disorder. In some embodiments, the composition is administered to treat a pancreatic cancer. In some embodiments, the composition is administered to treat metastatic pancreatic cancer.

In some embodiments, a pharmaceutical composition disclosed herein comprises a pharmaceutically acceptable carrier. In some embodiments, the pharmaceutical composition further comprises an adjuvant, excipient, preservative, agent for delaying absorption, filler, binder, adsorbent, buffer, disintegrating agent, and/or solubilizing agent.

In some embodiments, the pharmaceutical composition further comprises at least one pharmaceutically acceptable carrier. Suitable pharmaceutical carriers include inert diluents or fillers, water and/or various organic solvents.

In some embodiments, the composition includes a filler or diluent. In various embodiments, the filler or diluent is microcrystalline cellulose, silicified microcrystalline cellulose, lactose, mannitol, compressible sugar, calcium phosphate, calcium sulfate, calcium carbonate, calcium silicate and/or starch. In other embodiments, the filler or diluent is microcrystalline cellulose.

In some embodiments, the composition includes a disintegrant. In various embodiments, the disintegrant is croscarmellose sodium, sodium starch glycolate, crospovidone, methylcellulose, alginic acid, sodium alginate, starch derivatives, betonite and/or veegum. In some embodiment, the disintegrant is croscarmellose sodium.

In some embodiments, the composition includes a lubricant. In various embodiments, the lubricant is magnesium stearate, metallic stearates, talc, sodium stearyl fumarate and/or stearic acid. In some embodiments, the lubricant is magnesium stearate.

In some embodiments, the composition includes a wetting agent or surfactant. In various embodiments, the wetting agent or surfactant is sodium lauryl sulfate, glycerol, sorbitan oleates, sorbitan stearates, polyoxyethylenated sorbitan laurate, palmitate, stearate, oleate or hexaolate, polyoxyethylene stearyl alcohol and/or sorbitan monolaurate. In some embodiments, the wetting agent or surfactant is sodium lauryl sulfate.

Additional excipients (e.g., glidants, flavors, and/orcolorants) can also be added. For additional excipients see The Handbook of Pharmaceutical Excipients, 5^{th} Edition, 2005 and/orthe FDA Inactive Ingredient database.

In some embodiments, the composition comprises microcrystalline cellulose. In some embodiments, the composition comprises croscarmellose sodium. In some embodiments, the composition comprises sodium lauryl sulfate. In some embodiments, the composition comprises magnesium stearate.

In some embodiments, the composition further comprises a filler selected from microcrystalline cellulose, silicified microcrystalline cellulose, lactose, a compressible sugar, xylitol, sorbitol, mannitol, pregelatinized starch, maltodextrin, calcium phosphate, calcium carbonate, starch and/or a calcium silicate. In some embodiments, the composition further comprises a disintegrant selected from croscarmellose sodium, sodium starch glycolate, crospovidone, methylcellulose, alginic acid, sodium alginate, starch derivatives, betonite and/or veegum. In some embodiments, the composition further comprises a lubricant selected from magnesium stearate, metallic stearates, talc, sodium stearyl fumarate and/or stearic acid. In some embodiments, the composition further comprises a wetting agent or surfactant selected from sodium lauryl sulfate, glycerol, sorbitan oleates, sorbitan stearates, polyoxyethylenated sorbitan laurate, palmitate, stearate, oleate or hexaolate, polyoxyethylene stearyl alcohol and/or sorbitan monolaurate.

Dosage Forms

In some embodiments, a composition disclosed herein is formulated for oral administration. In some embodiments, a composition disclosed herein is administered as a tablet, capsule, pill, powder, solution, suspension, a gel cap, a caplet, a pellet, or a bead.

In some embodiments, a compositing disclosed herein is administered via a tablet. In some embodiments, a tablet comprises an inert diluent (e.g., calcium carbonate, sodium carbonate, lactose, calcium phosphate or sodium phosphate); a granulating and/or disintegrating agent (e.g., croscarmellose sodium, crospovidone or sodium starch glycolate); a filler (e.g., microcrystalline cellulose, silicified microcrystalline cellulose, pregelatinized starch, lactose, dicalcium phosphate, or compressible sugar); a binder (e.g., hypromellose, povidone, starch, gelatin, polyvinyl-pyrrolidone, or acacia); a surfactant (e.g., sodium lauryl sulfate) and/or a lubricant and/or processing aide (e.g., talc, sodium croscarmellose, corn starch, or alginic acid, magnesium stearate, stearic acid, colloidal silicion dioxide, and/or sodium lauryl sulfate). In some embodiments, a tablet further comprises a sweetening agent, a flavoring agent, a coloring agent and/or a preserving agent.

In some embodiments, a tablet comprises citric acid, a disintegrant (e.g., starch, alginic acid and/orcertain complex silicates), and/or a binding agent (e.g., sucrose, gelatin and/oracacia).

In some embodiments, the tablet is un-coated or coated. In certain instances, a coating masks the taste of a composition. In certain instances, a coating modifies disintegration and/or absorption in the gastrointestinal tract.

In some embodiments, a tablet disclosed herein is prepared according to any suitable method. In some embodiments, a tablet disclosed herein is prepared by dry blending. In some embodiments, a compound disclosed herein is incorporated into the dosage form by dry blending with an excipient followed by compression into a tablet form. In some embodiments, a compressed tablet is prepared by compressing in a suitable machine the active ingredient in a free-flowing form (e.g., a powder or granules), optionally mixed with a binder, an inert diluent, and/or a lubricating, surface active or dispersing agent.

In some embodiments, a tablet disclosed herein is prepared according to any suitable method. In some embodiments, a tablet disclosed herein is prepared by wet granulation. In some embodiments, a compound disclosed herein is added to the dry excipients and mixed prior to the addition of the binder solution, or the drug substance is dissolved and added as a solution as part of granulation. In the wet granulation technique the surfactant, if used, is added to the dry excipients or added to the binder solution and incorporated in a solution form.

In some embodiments, a compositing disclosed herein is administered via a capsule. In some embodiments, the capsule is a hard capsule. In some embodiments, the active ingredient is mixed with an inert solid diluent, for example, calcium carbonate, calcium phosphate or kaolin. In some embodiments, the capsule is a soft capsule. In some embodiments, the active ingredient is mixed with water soluble carrier such as polyethyleneglycol or an oil medium, for example peanut oil, liquid paraffin, or olive oil.

In some embodiments, a capsule disclosed herein is prepared according to any suitable method. In some embodiments, a compound disclosed herein is dissolved in a material (e.g., a molten form of a high molecular weight polyethylene glycol) that is filled into a hard gelatin capsule shell that is subsequently banded and sealed. In some embodiments, a compound disclosed herein is dissolved a molten form of a high molecular weight polyethylene glycol. In some embodiments, the mixture is cooled and then filled into a gelatin capsule.

In some embodiments, the composition is in the form of a capsule or tablet and/or has a total weight of about 50 mg to about 1000 mg. In some embodiments, the composition is in the form of a capsule or tablet and/or has a total weight selected from the group consisting of 50 mg, 75mg, 100 mg, 150 mg, 200 mg, 250 mg, 300 mg, 350 mg, 400 mg, 450 mg, and/or500 mg. In some embodiments, the composition is in the form of a capsule or tablet and/or has a total weight of about 240 mg.

In some embodiments, the composition is in the form of a capsule or tablet and the dosage form comprises from about 1 to about 50 mg of a compound disclosed herein, having a USP acceptance value for content uniformity of less than about 15.

In some embodiments, a compound disclosed herein is administered as an aqueous suspension. In some embodiments, an aqueous suspension comprises a sweetening or flavoring agent, coloring matters or dyes and, if desired, emulsifying agents or suspending agents, together with diluents water, ethanol, propylene glycol, glycerin, or combinations thereof. In some embodiments, an aqueous suspension comprises a suspending agent. In some embodiments, an aqueous suspension comprises sodium carboxymethylcellulose, methylcellulose, hydroxypropylmethyl-cellulose, sodium alginate, polyvinyl-pyrrolidone, gum tragacanth and/or gum acacia. In some embodiments, an aqueous suspension comprises a dispersing or wetting agent. In some embodiments, an aqueous suspension comprises a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethylene-oxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. In some embodiments, an aqueous suspension comprises a preservative. In some embodiments, an aqueous suspension comprises ethyl, or n-propyl p-hydroxybenzoate. , In some embodiments, an aqueous suspension comprises a sweetening agent. In some embodiments, an aqueous suspension comprises sucrose, saccharin or aspartame.

In some embodiments, a compound disclosed herein is administered as an oily suspension. In some embodiments, an oily suspension is formulated by suspending the active ingredient in a vegetable oil (e.g., arachis oil, olive oil, sesame oil or coconut oil), or in mineral oil (e.g., liquid paraffin). In some embodiments, an oily suspension comprises a thickening agent (e.g., beeswax, hard paraffin or cetyl alcohol). In some embodiments, an oily suspension comprises sweetening agents (e.g., those set forth above). In some embodiments, an oily suspension comprises an anti-oxidant (e.g., butylated hydroxyanisol or alpha-tocopherol).

In some embodiments, a composition disclosed herein is formulated for parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural and/or subcutaneous). In some embodiments, a composition disclosed herein is administered as a sterile solution, suspension or emulsion.

In some embodiments, a formulation for parenteral administration includes aqueous and/or non-aqueous (oily) sterile injection solutions of the active compounds which may contain antioxidants, buffers, bacteriostats and/or solutes which render the formulation isotonic with the blood of the intended recipient; and/or aqueous and/or non-aqueous sterile suspensions which may include a suspending agent and/or a thickening agent. In some embodiments, a formulation for parenteral administration includes suitable stabilizers or agents which increase the solubility of the compounds to allow for the preparation of highly concentrated solutions.

In some embodiments, a compound disclosed herein is administered as an aqueous suspension. In some embodiments, an aqueous suspension comprises water, Ringer's solution and/or isotonic sodium chloride solution.

In some embodiments, a compound disclosed herein is administered as an oil-in-water microemulsion where the active ingredient is dissolved in the oily phase. In some embodiments, a compound disclosed herein is dissolved in a fatty oil (e.g., sesame oil, or synthetic fatty acid esters, (e.g., ethyl oleate or triglycerides, or liposomes. In some embodiments, a compound disclosed herein is dissolved in a mixture of soybean oil andrlecithin. In some embodiments, the oil solution is introduced into a water and glycerol mixture and processed to form a microemulsion.

In some embodiments, a composition formulated for parenteral administration is administered as a single bolus shot. In some embodiments, a composition formulated for parenteral administration is administered via a continuous intravenous delivery device (e.g., Deltec CADD-PLUS™ model 5400 intravenous pump).

In some embodiments, a formulation for injection is presented in unit dosage form, e.g., in ampoules or in multi-dose containers, with an added preservative. In some embodiments, a formulation for injection is stored in powder form or in a freeze-dried (lyophilized) condition requiring only the addition of the sterile liquid carrier, for example, saline or sterile pyrogen-free water, immediately prior to use.

In some embodiments, a formulation disclosed herein is administered by depot preparation. In some embodiments, a depot preparation is administered by implantation (for example subcutaneously or intramuscularly) or by intramuscular injection.

In some embodiments, a composition disclosed herein is formulated for topical administration. As used herein, topical administration means application of a composition such that the compound does not significantly enter the blood stream. In some embodiments, a composition disclosed herein is applied to the epidermis, the buccal cavity, the ear, eye and/or nose.

In some embodiments, a composition formulated for topical administration is formulated as a gel, liniment, lotion, cream, ointment or paste, solution, suspension, emulsion, or powder. In some embodiments, a composition disclosed herein is administered as an ointment or cream. In some embodiments, a composition disclosed herein is administered as a mouth wash. In some embodiments, a composition disclosed herein is administered via inhalation.

In some embodiments, a composition formulated for administration via inhalation is delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount.

Alternatively, for administration by inhalation or insufflation, pharmaceutical preparations may take the form of a dry powder composition, for example a powder mix of the compound and a suitable powder base such as lactose or starch. The powder composition is presented in unit dosage form, in for example, capsules, cartridges, gelatin or blister packs from which the powder is administered with the aid of an inhalator or insufflator. For buccal or sublingual administration, the compositions may take the form of tablets, lozenges, pastilles, or gels formulated in conventional manner. Such compositions may comprise the active ingredient in a flavored basis such as sucrose and acacia or tragacanth.

In some embodiments, a composition disclosed herein is formulated for rectal administration. In some embodiments, a composition disclosed herein is administered as a suppository. In some embodiments, a composition suitable for rectal administration is prepared by mixing a compound disclosed herein with a suitable non-irritating excipient which is solid at ordinary temperatures but liquid at the rectal temperature and will therefore melt in the rectum to release the drug. In some embodiments, a composition suitable for rectal administration is prepared by mixing a compound disclosed herein with cocoa butter, glycerinated gelatin, hydrogenated vegetable oils, mixtures of polyethylene glycols of various molecular weights or fatty acid esters of polyethylene glycol.

For methods of preparing various pharmaceutical compositions see Remington's Pharmaceutical Sciences, Mack Publishing Company, Ester, Pa., 18th Edition (1990).

In some embodiments, the dosage form releases at least 60 percent of the drug within 30 minutes using U.S. Pharmacopeia (USP) Apparatus II at 50 rpm with 1% sodium lauryl sulfate in water as the dissolution medium. In some embodiments, the dosage form releases about 60-100 percent of the drug within 30 minutes using U.S. Pharmacopeia (USP) Apparatus II at 50 rpm with 1% sodium lauryl sulfate in water as the dissolution medium. In some embodiments, the dosage form releases about 60-90 percent of the drug within 30 minutes using U.S. Pharmacopeia (USP) Apparatus II at 50 rpm with 1% sodium lauryl sulfate in water as the dissolution medium. In some embodiments, the dosage form releases about 60-80 percent of the drug within 30 minutes using U.S. Pharmacopeia (USP) Apparatus II at 50 rpm with 1% sodium lauryl sulfate in water as the dissolution medium.

Dosages

The amount of pharmaceutical compositions administered will firstly be dependent on the mammal being treated. In the instances where pharmaceutical compositions are administered to a human subject, the daily dosage will normally be determined by the prescribing physician with the dosage generally varying according to the age, sex, diet, weight, general health and response of the individual, the severity of the individual's symptoms, the precise indication or condition being treated, the severity of the indication or condition being treated, time of administration, route of administration, the disposition of the composition, rate of excretion, drug combination, and the discretion of the prescribing physician.

In some embodiments, the dose is sufficient to result in slowing, and/or regressing, the growth of the tumors and/or causing complete regression of the cancer. Regression of a tumor in a patient is measured with reference to the diameter of a tumor. Decrease in the diameter of a tumor indicates regression. Regression is also indicated by failure of tumors to reoccur after treatment has stopped.

In some embodiments, the therapeutically effective amount is estimated initially either in cell culture assays, e.g., of neoplastic cells, or in animal models, usually rats, mice, rabbits, dogs, or pigs. In some embodiments, the animal model is used to determine the appropriate concentration range and route of administration. Such information can then be used to determine useful doses and routes for administration in humans. Therapeutic/prophylactic efficacy and toxicity is determined by standard pharmaceutical procedures in cell cultures or experimental animals, e.g., ED₅₀ (the dose therapeutically effective in 50% of the population) and LD₅₀ (the dose lethal to 50% of the population). The dose ratio between therapeutic and toxic effects is the therapeutic index, and it is expressed as the ratio, ED₅₀/LD₅₀. The dosage may vary within this range depending upon the dosage form employed, sensitivity of the patient, and the route of administration.

Dosage and administration are adjusted to provide sufficient levels of the active agent(s) or to maintain the desired effect. Factors which is taken into account include the severity of the disease state, general health of the subject, age, weight, and gender of the subject, diet, time and frequency of administration, drug combination(s), reaction sensitivities, and tolerance/response to therapy. In some embodiments, pharmaceutical compositions is administered every 3 to 4 days, every week, or once every two weeks depending on half-life and clearance rate of the particular formulation.

Dosages of the pharmaceutical compositions may vary depending on the agent, the age, weight, and clinical condition of the recipient patient, and the experience and judgment of the clinician or practitioner administering the therapy. Generally, the dose should be sufficient to result in slowing, and/or regressing, the growth of the tumors and/or causing complete regression of the cancer. Dosages can range from about 0.01 mg/kg per day to about 3000 mg/kg per day. In some embodiments, dosages can range from about 1 mg/kg per day to about 1000 mg/kg per day. In an aspect, the dose will be in the range of about 0.1 mg/day to about 70 g/day; about 0.1 mg/day to about 25 g/day; about 0.1 mg/day to about 10 g/day; about 0.1 mg to about 3g/day; or about 0.1 mg to about 1 g/day, in single, divided, or continuous doses (which dose is adjusted for the patient's weight, body surface, and age). An effective amount of a pharmaceutical agent is that which provides an objectively identifiable improvement as noted by the clinician or other qualified observer. For example, regression of a tumor in a patient is measured with reference to the diameter of a tumor. Decrease in the diameter of a tumor indicates regression. Regression is also indicated by failure of tumors to reoccur after treatment has stopped. As used herein, the term “dosage effective manner” refers to amount of an active compound to produce the desired biological effect in a subject or cell.

Disclosed herein, in certain embodiments, is a method of treating a proliferative disorder, comprising administering to an individual in need thereof a therapeutically effective amount of a pharmaceutical composition comprising (S)-N-(3,4-difluoro-2-(2-fluoro-4-iodophenylamino)-6-methoxyphenyl)-1-(2,3-dihydroxypropyl)cyclopropane-1-sulfonamide; N-(4-(2-fluoro-4-iodophenylamino)-1,5-dimethyl-6-oxo-1,6-dihydropyridin-3-yl)cyclopropanesulfonamide; pharmaceutically acceptable salts of either compound; polymorphs of either compound (see e.g., U.S. patent application Ser. No. 12/399,848); or combinations thereof and a pharmaceutically acceptable carrier. In some embodiments, the composition maintains a plasma concentration of about 0.15 μM to about 50 μM and treats the proliferative disorder. In some embodiments, the plasma concentration is about 0.1 μM to about 100 μM, about 0.125 μM to about 75 μM; about 0.15 μM to about 50 μM; about 0.175 μM to about 30 μM; and about 0.2 μM to about 20 μM. In some embodiments, the pharmaceutical composition maintains a suitable plasma concentration for at least a month, at least a week, at least 24 hours, at least 12 hrs, at least 6 hrs, at least 1 hour. In some embodiments, a suitable plasma concentration of the pharmaceutical composition is maintained indefinitely.

In some embodiments, the composition has an AUC (area under the curve) range of about 0.5 μM-hr to about 100 μM-hr, about 0.5 μM-hr to about 50 μM-hr, about 1 μM-hr to about 25 μM-hr, about 1 μM-hr to about 10 μM-hr; about 1.25 μM-hr to about 6.75 μM-hr, about 1.5 μM-hr to about 6.5 μM-hr.

In some embodiments, the composition is administered at a dosage from about 2 mg/m²to 5000 mg/m² per day, from about 20 mg/m2 to 2000 mg/m² per day, from about 20 mg/m² to 500 mg/m² per day, from about 30 to 300 mg/m per day. In some embodiments, 2 mg/m² to 5000 mg/m² per day is the administered dosage for a human. In some embodiments, the pharmaceutical composition is administered at a dosage from about 10 to 1,000,000 μg per kilogram body weight of recipient per day; about 100 to 500,000 μg per kilogram body weight of recipient per day, from about 1000 to 250,000 μg per kilogram body weight of recipient per day, from about 10,000 to 150,000 μg per kilogram body weight of recipient per day.

In some embodiments, the amount of compound disclosed herein is administered in a single dose, once daily. In some embodiments, the amount of compound disclosed herein is administered in multiple doses, more than once per day. In some embodiments, the amount of compound disclosed herein is administered twice daily. In some embodiments, the amount of compound disclosed herein is administered three times per day. In some embodiments, the amount of compound disclosed herein is administered four times per day. In some embodiments, the amount of compound disclosed herein is administered more than four times per day.

In some instances, dosage levels below the lower limit of the aforesaid range is more than adequate, while in other cases still larger doses is employed without causing any harmful side effect, e.g. by dividing such larger doses into several small doses for administration throughout the day. The amount administered will vary depending on the particular IC₅₀ value of the compound used. In combinational applications in which the compound is not the sole therapy, it is possible to administer lesser amounts of compound and still have therapeutic or prophylactic effect.

Combination Therapies

In some embodiments, a compound disclosed herein is administered in combination with a second therapeutic agent. In some embodiments, a compound disclosed herein is administered in combination with surgery, and/or radiation therapy.

In some embodiments, the second therapeutic agent is selected from cytotoxic agents, anti-angiogenesis agents and/or anti-neoplastic agents. In some embodiments, the second therapeutic agent is selected from alkylating agents, anti-metabolites, epidophyllotoxins; antineoplastic enzymes, topoisomerase inhibitors, procarbazines, mitoxantrones, platinum coordination complexes, biological response modifiers and growth inhibitors, hormonal/anti-hormonal therapeutic agents, haematopoietic growth factors, aromatase inhibitors, anti-estrogens, anti-androgens, corticosteroids, gonadorelin agonists, microtubule active agents, nitrosoureas, lipid or protein kinase targeting agents, IMiDs, protein or lipid phosphatase targeting agents, anti-angiogenic agents, Akt inhibitors, IGF-I inhibitors, FGF3 modulators, mTOR inhibitors, Smac mimetics, HDAC inhibitors, agents that induce cell differentiation, bradykinin 1 receptor antagonists, angiotensin II antagonists, cyclooxygenase inhibitors, heparanase inhibitors, lymphokine inhibitors, cytokine inhibitors, IKK inhibitors, P38MAPK inhibitors, HSP90 inhibitors, multlikinase inhibitors, bisphosphanate, rapamycin derivatives, anti-apoptotic pathway inhibitors, apoptotic pathway agonists, PPAR agonists, RAR agonists, inhibitors of Ras isoforms, telomerase inhibitors, protease inhibitors, metalloproteinase inhibitors, aminopeptidase inhibitors, SHIP activators—AQX-MN100, Humax-CD20 (ofatumumab), CD20 antagonists, IL2-diptheria toxin fusions, or combinations thereof.

In additional aspects, (S)-N-(3,4-difluoro-2-(2-fluoro-4-iodophenylamino)-6-methoxyphenyl)-1-(2,3-dihydroxypropyl)cyclopropane-1-sulfonamide or a pharmaceutically acceptable salt thereof or N-(4-(2-fluoro-4-iodophenylamino)-1,5-dimethyl-6-oxo-1,6-dihydropyridin-3-yl)cyclopropanesulfonamide or a pharmaceutically acceptable salt thereof is administered in combination with a chemotherapeutic agent. Exemplary chemotherapeutics with activity against cell proliferative disorders, such as pancreatic cancer, are known to those of ordinary skill in the art, and is found in reference texts such as the Physician's Desk Reference, 59th Edition, Thomson PDR (2005). Examples of chemotherapeutic agents include, but are not limited to a taxane, an aromatase inhibitor, an anthracycline, a microtubule targeting drug, a topoisomerase poison drug, a targeted monoclonal or polyclonal antibody, an inhibitor of a molecular target or enzyme (e.g., a kinase inhibitor), or a cytidine analogue drug. Examples of chemotherapeutic agents include, but are not limited to, tamoxifen, raloxifene, anastrozole, exemestane, letrozole, trastuzumab, imatanib, paclitaxel, gefitinib, erlotinib, cyclophosphamide, lovastatin, minosine, araC, 5-fluorouracil (5-FU), methotrexate (MTX), docetaxel, goserelin, bevacizumab, vincristin, vinblastin, nocodazole, teniposide, etoposide, epothilone, navelbine, camptothecin, daunonibicin, dactinomycin, mitoxantrone, amsacrine, doxorubicin adriamycin, epirubicin or idarubicin. In some embodiments, the chemotherapeutic agent is a cytokine such as G-CSF (granulocyte colony stimulating factor). In some embodiments, (S)-N-(3,4-difluoro-2-(2-fluoro-4-iodophenylamino)-6-methoxyphenyl)-1-(2,3-dihydroxypropyl)cyclopropane-1-sulfonamide or a pharmaceutically acceptable salt thereof or N-(4-(2-fluoro-4-iodophenylamino)-1,5-dimethyl-6-oxo-1,6-dihydropyridin-3-yl)cyclopropanesulfonamide or a pharmaceutically acceptable salt thereof is administered in combination with radiation therapy. In yet another aspect, (S)-N-(3,4-difluoro-2-(2-fluoro-4-iodophenylamino)-6-methoxyphenyl)-1-(2,3-dihydroxypropyl)cyclopropane-1-sulfonamide or a pharmaceutically acceptable salt thereof or N-(4-(2-fluoro-4-iodophenylamino)-1,5-dimethyl-6-oxo-1,6-dihydropyridin-3-yl)cyclopropanesulfonamide or a pharmaceutically acceptable salt thereof is administered in combination with standard chemotherapy combinations such as, but not limited to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), or CMFP (cyclophosphamide, methotrexate, 5-fluorouracil and prednisone).

In some embodiments, combination therapy includes administering a compound described herein with taxol; a compound as described herein with docetaxel; a compound described herein with vincristin; a compound described herein with vinblastin; a compound described herein with nocodazole; a compound described herein with teniposide; a compound described herein with etoposide; a compound described herein with adriamycin; a compound described herein with epothilone; a compound described herein with navelbine; a compound described herein with camptothecin; a compound described herein with daunorubicin; a compound described herein with dactinomycin; a compound described herein with mitoxantrone; a compound described herein with amsacrine; a compound described herein with epirubicin; or a compound described herein with idarubicin. In another red aspect, combination therapy includes a compound described herein with gemcitabine.

The combination therapy agents described herein is administered singly and sequentially, or in a cocktail or combination containing both agents or one of the agents with other therapeutic agents, including but not limited to, immunosuppressive agents, potentiators and side-effect relieving agents.

Kits

The compounds, compositions and/or methods described herein provide kits for the treatment of disorders, (e.g., the ones described herein). These kits comprise a compound, compounds or compositions described herein in a container and, optionally, instructions teaching the use of the kit according to the various methods and/or approaches described herein. Such kits may also include information, (e.g., scientific literature references, package insert materials, clinical trial results, and/or summaries of these and/or the like), which indicate or establish the activities and/or advantages of the composition, and/or which describe dosing, administration, side effects, drug interactions, or other information useful to the health care provider. Such information is based on the results of various studies, for example, studies using experimental animals involving in vivo models and/or studies based on human clinical trials. Kits described herein is provided, marketed and/or promoted to health providers, including physicians, nurses, pharmacists, formulary officials, and/or the like. Kits may also, in some embodiments, be marketed directly to the consumer.

### EXAMPLES

For simplicity the following abbreviations is used :

| Compound Name | Compound Structure | Abbreviated to: |
|---|---|---|
| (S)-N-(3,4-difluoro-2-(2-fluoro-4- iodophenylamino)-6-methoxyphenyl)-1- (2,3-dihydroxypropyl)cyclopropane-1- sulfonamide | 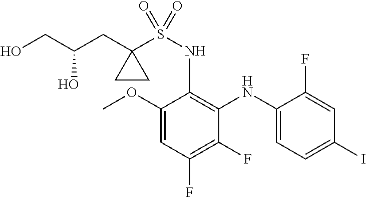 | Compound A |
| N-(4-(2-fluoro-4-iodophenylamino)-1,5- dimethyl-6-oxo-1,6-dihydropyridin-3- yl)cyclopropanesulfonamide | 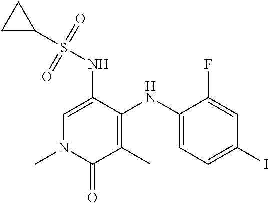 | Compound B |

I In vitro activity

### Example 1

The in vitro activity of compound A was determined in the human pancreatic cancer cell line BxPC3 (normal BTAF status). EC₅₀ values were determined (in 1% FBS and with 45 mg/mL has), as follows

EC₅₀ (nM)
(±standard deviation)

| 1% FBS | +45 mg/mL hSA |
|---|---|
| 15.8 ± 2.4 | 207 ± 29 |

### Example 2

The in vitro activity of compound A is determined in the pancreatic cancer cell line MIA-PaCa-2.

The in vitro activity of compound A is determined in the pancreatic cancer cell line Panc-1.

The in vitro activity of compound A is determined in the pancreatic cancer cell line AsPC-1.

The in vitro activity of compound A is determined in the pancreatic cancer cell line BxPC-3.

The in vitro activity of compound A is determined in the pancreatic cancer cell line SU.86.86.

The in vitro activity of compound A is determined in the pancreatic cancer cell line CFPAC-1.

The in vitro activity of compound A is determined in the pancreatic cancer cell line HPAF-II.

The in vitro activity of compound A is determined in the pancreatic cancer cell line HPAC.

The in vitro activity of compound A is determined in the pancreatic cancer cell line SW 1990.

The in vitro activity of compound A is determined in the pancreatic cancer cell line Panc 10.05.

The in vitro activity of compound A is determined in the pancreatic cancer cell line Panc 03.27.

The in vitro activity of compound A is determined in the pancreatic cancer cell line Panc 08.13.

The in vitro activity of compound A is determined in the pancreatic cancer cell line Panc 02.03.

The in vitro activity of compound A is determined in the pancreatic cancer cell line Panc 02.13.

The in vitro activity of compound A is determined in the pancreatic cancer cell line Panc 04.03.

The in vitro activity of compound A is determined in the pancreatic cancer cell line Panc 05.04.

The in vitro activity of compound A is determined in the pancreatic cancer cell line PL45.

The in vitro activity of compound A is determined in the pancreatic cancer cell line Capan-1.

The in vitro activity of compound A is determined in the pancreatic cancer cell line Hs766T.

### Example 3

The in vitro activity of compound B is determined in the pancreatic cancer cell line MIA-PaCa-2.

The in vitro activity of compound B is determined in the pancreatic cancer cell line Panc-1.

The in vitro activity of compound B is determined in the pancreatic cancer cell line AsPC-1.

The in vitro activity of compound B is determined in the pancreatic cancer cell line BxPC-3.

The in vitro activity of compound B is determined in the pancreatic cancer cell line SU.86.86.

The in vitro activity of compound B is determined in the pancreatic cancer cell line CFPAC-1.

The in vitro activity of compound B is determined in the pancreatic cancer cell line HPAF-II.

The in vitro activity of compound B is determined in the pancreatic cancer cell line HPAC.

The in vitro activity of compound B is determined in the pancreatic cancer cell line SW 1990.

The in vitro activity of compound B is determined in the pancreatic cancer cell line Panc 10.05.

The in vitro activity of compound B is determined in the pancreatic cancer cell line Panc 03.27.

The in vitro activity of compound B is determined in the pancreatic cancer cell line Panc 08.13.

The in vitro activity of compound B is determined in the pancreatic cancer cell line Panc 02.03.

The in vitro activity of compound B is determined in the pancreatic cancer cell line Panc 02.13.

The in vitro activity of compound B is determined in the pancreatic cancer cell line Panc 04.03.

The in vitro activity of compound B is determined in the pancreatic cancer cell line Panc 05.04.

The in vitro activity of compound B is determined in the pancreatic cancer cell line PL45.

The in vitro activity of compound B is determined in the pancreatic cancer cell line Capan-1.

The in vitro activity of compound B is determined in the pancreatic cancer cell line Hs766T.

II In vivo activity

### Example 4

In vivo activity of compound B: Bx-PC3-e242 Xenograft Study Bx-PC3-e242 cells were injected into 11-week-old female (nu/nu) mice. Tumors were allowed to reach 115.5-116.7 mm³ (group mean tumor range); 63-196 mm³ (Individual Tumor Range) in size (23 days) and mice were randomized into 6 groups of 9 animals (body weight range 18.0-25.2 g). Mice were treated by according to the chart below:

| | | Treatment Regimen 1 | | | | Treatment Regimen 2 | | | |
|---|---|---|---|---|---|---|---|---|---|
| Grp | n | Agent | Mg/kg | Route | Schedule | Agent | Mg/kg | Route | Schedule |
| 1 | 9 | Vehicle | — | po | qd × 14 | — | — | — | — |
| 2 | 9 | Control | 30 | iv | qod × 5 | — | — | — | — |
| 3 | 9 | Cmpd B | 25 | po | qd × 14 | — | — | — | — |
| 4 | 9 | Cmpd B | 6.25 | po | bid × 14 | — | — | — | — |
| | | | | | first day 1 | | | | |
| | | | | | dose | | | | |
| 5 | 9 | Cmpd B | 9 | po | | — | — | — | — |
| 6 | 9 | Cmpd B | 12.5 | po | | Cmpd B | 6.25 | po | bid × 11 first |
| | | | | | | | | | day 1 dose |
| | | | | | | | | | (start on day 4) |

Tumor Measurement

Tumors were measured with a caliper and tumor volumes calculated using the following formula:

$$Tumor volume ( {mm}^{3} ) = \frac{{w}^{2} xl}{2}$$

where w=width and l=length in mm of a tumor. Tumor weight is estimated with the assumption that 1 mg is equivalent to 1 mm³ of tumor volume.

Interim Median Tumor Volumes are shown in the table below :

| Group | D1 | D5 | D10 | D15 | D22 | D29 | D36 | D43 | D50 |
|---|---|---|---|---|---|---|---|---|---|
| Group 1 | 108 (9) | 196 (9) | 864 (9) | 1090 (8) | 1008 (3) | 726 (1) | 1099 (1) | — | — |
| Group 2 | 88 (9) | 88 (9) | 144 (9) | 40 (9) | 108 (9) | 288 (9) | 525 (8) | 1183 (5) | 1009 (2) |
| Group 3 | 108 (9) | 75 (9) | 196 (9) | 196 (9) | 446 (9) | 864 (5) | 867 (4) | 1015 (2) | 1327 (2) |
| Group 4: | 108 (9) | 75 (9) | 196 (9) | 126 (9) | 288 (9) | 509 (8) | 600 (6) | 1268 (3) | 976 (2) |
| Group 5 | 108 (9) | 63 (9) | 172 (9) | 126 (9) | 288 (9) | 566 (8) | 600 (5) | 1008 (1) | 1470 (1) |
| Group 6 | 75 (9) | 75 (9) | 259 (9) | 172 (9) | 550 (9) | 1008 (9) | 900 (2) | 1470 (1) | — |

* Weekly Median Tumor Volume = median tumor volume (mm³) of animals on given day (includes animals with tumor volume at endpoint), number of animals in parentheses

A Summary of various Interim Responses are shown in the table below :

| | MTV (n) | Median | | | MTV (n) | | No. of | Mean BW | No. of |
|---|---|---|---|---|---|---|---|---|---|
| Group | D54 | TTE | T − C | % TGD | D15 | % TGI | PR | Nadir | NTR |
| Group 1 | — | 19.5 | — | — | 1099 (9) | — | 0 | — | 0 |
| Group 2 | 726 (1) | 45.5 | 26.0 | 133% | 40 (9) | 96% | 1 | −3.6% Day 12 | 0 |
| Group 3 | 1470 (1) | 31.6 | 12.1 | 62% | 196 (9) | 82% | 0 | — | 0 |
| Group 4: | 486 (1) | 37.3 | 17.8 | 91% | 126 (9) | 89% | 0 | — | 1 |
| Group 5 | — | 37.8 | 18.3 | 94% | 126 (9) | 89% | 0 | — | 0 |
| Group 6 | — | 32.9 | 13.4 | 69% | 172 (9) | 84% | 0 | — | 0 |

MTV (n) = median tumor volume (mm³) for the number of animals on the day of TGD analysis (excludes animals reaching endpoint)
TTE = time to endpoint;
T − C = difference between median TTE (days) of treated versus control group;
% TGD = [(T − C)/C] × 100
MTV (n) = median tumor volume (mm³) for the number of animals on the day of TGI analysis (includes animals with tumor volume at endpoint)
% TGI = [1 − (T/C)] × 100 = Percent tumor growth inhibition, compared to Group 1
PR = partial regression
Mean BW Nadir = lowest group mean body weight, as % change from Day 1;
— indicates no decrease in mean body weight was observed
NTR = non-treatment-related death
No complete regression observed
No treatment-related deaths observed

Endpoint Determination

Each animal was euthanized when the tumor reached endpoint size or at the end of the study, whichever comes first. The time to endpoint (TTE) for each mouse is calculated according to the following equation

$$T T E ( days ) = \frac{{log}_{10} ( endpoint volume , {mm}^{2} ) - b}{m}$$

where b is the intercept.

The study endpoint was 1500 mm³, with a study duration of 54 days.

Regressions

Treatment may cause partial regression (PR) or complete regression (CR) of the tumor in an animal. In a PR response, the tumor volume is 50% or less of its Day 1 volume for three consecutive measurements during the course of the study.

TGD Analysis

Treatment outcome is evaluated by tumor growth delay (TGD), defined as the increase in the median time to endpoint (TTE) in a treatment group compared to the control group:

TGD=T−C

expressed in days, or as a percentage of the median TTE

% TGD=(T−C)/C×100

Individual TTE values are shown below:

| 1500 mm³ | Group 1 | Group 2 | Group 3 | Group 4 | Group 5 | Group 6 |
|---|---|---|---|---|---|---|
| 1 | 25.6 | 38.3 | 28.3 | 51.4 | 42.8 | 45.6 |
| 2 | 23.5 | 38.5 | 54.0 | 50.0 | 33.8 | 35.7 |
| 3 | 38.8 | 54.0 | 50.1 | 35.1 | 41.7 | 29.7 |
| 4 | 13.7 | 48.8 | 26.5 | 36.9 | 42.8 | 33.3 |
| 5 | 19.5 | 52.4 | 31.6 | *40.0 | 50.3 | 41.0 |
| 6 | 18.3 | 49.0 | 38.2 | 29.0 | 30.5 | 31.1 |
| 7 | 16.3 | 34.1 | 40.2 | 37.7 | 28.2 | 31.8 |
| 8 | 15.9 | 45.5 | 26.8 | 31.5 | 37.8 | 32.8 |
| 9 | 20.1 | 40.4 | 23.4 | 54.0 | 31.8 | 32.9 |
| Median TTE: | 19.5 | 45.5 | 31.6 | 37.3 | 37.8 | 32.9 |
| Mean TTE: | 21.3 | 44.6 | 35.4 | 40.7 | 37.8 | 34.9 |
| Mean TV(n): | — | 1196 | 1635 | 1076 | 1913 | — |
| | | (2) | (2) | (2) | (1) | |

*Non-treatment-related death due to unknown etiology

TGI Analysis

Response to treatment was also evaluated for tumor growth inhibition (TGI), defined as the difference between the median tumor volumes (MTVs) of treated and control mice.

$$\% T G I = \frac{Median Tumor {Volume}_{control} - Median Tumor {Volume}_{treated}}{Median Tumor {Volume}_{control}} \times 100$$

Toxicity

Animals were weighed daily for the first five days of the study and then twice weekly. The mice are observed frequently for overt signs of any adverse, treatment-related side effects, and clinical signs of toxicity were recorded when observed.

Graphical Analyses

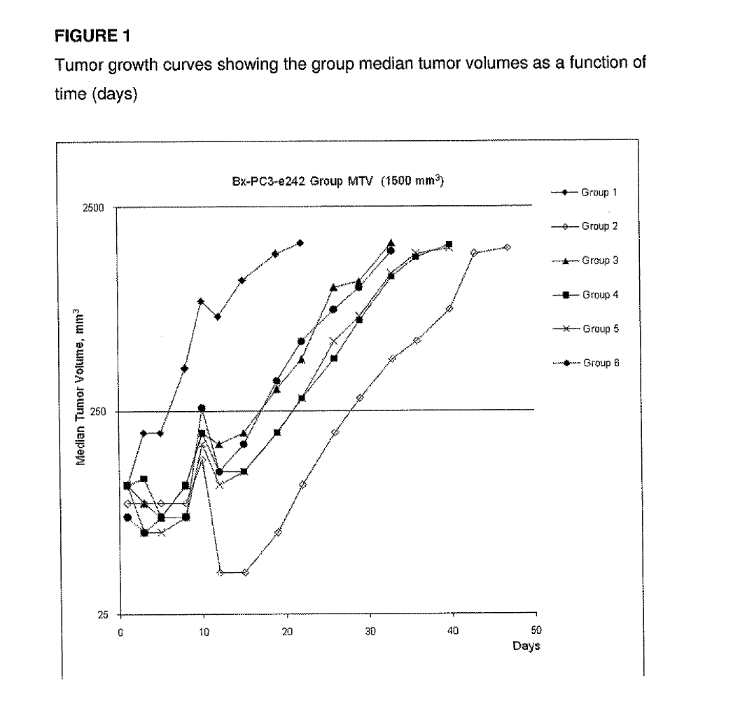

Tumor growth curves showing the group median tumor volumes as a function of time (days) as presented in FIG. 1.

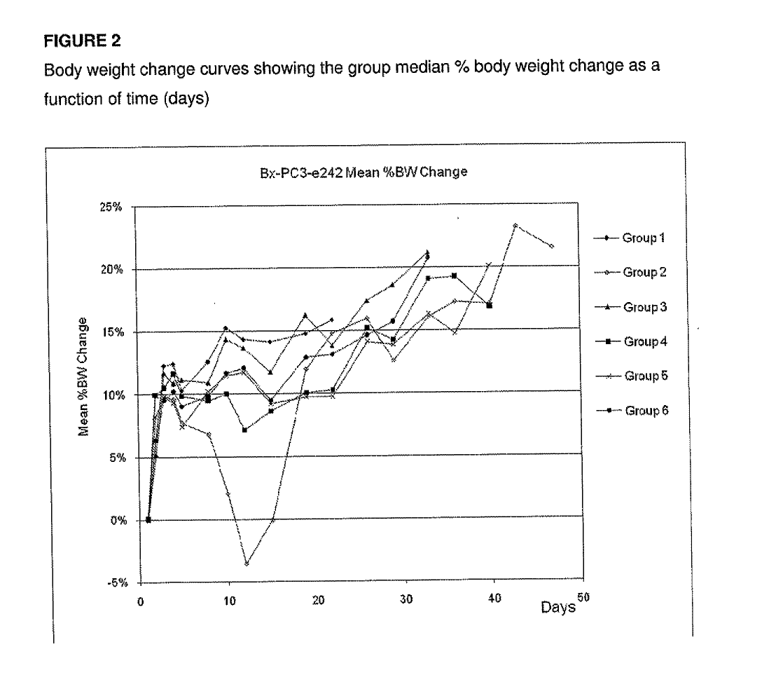

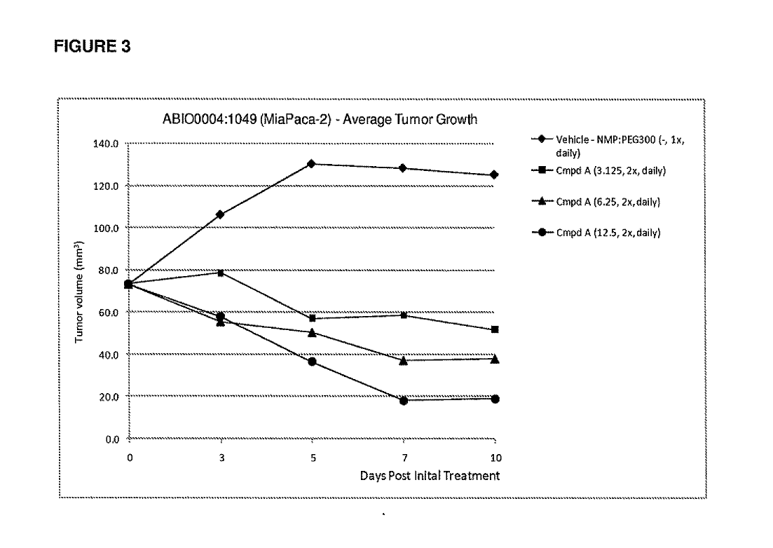

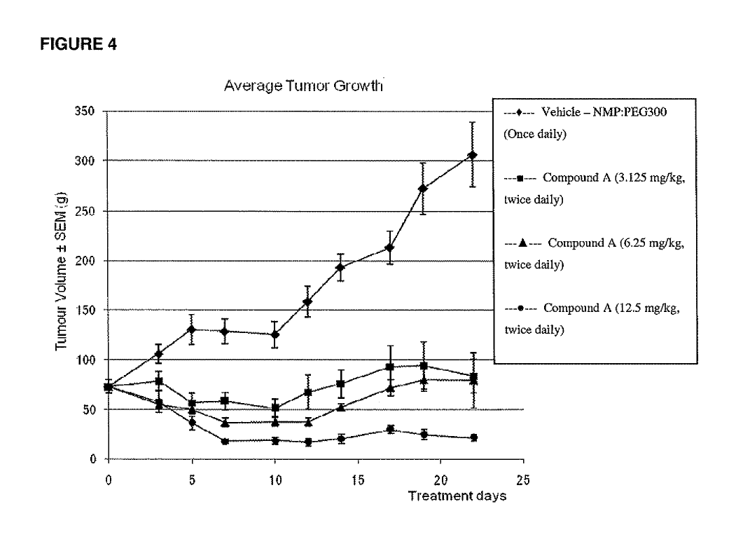

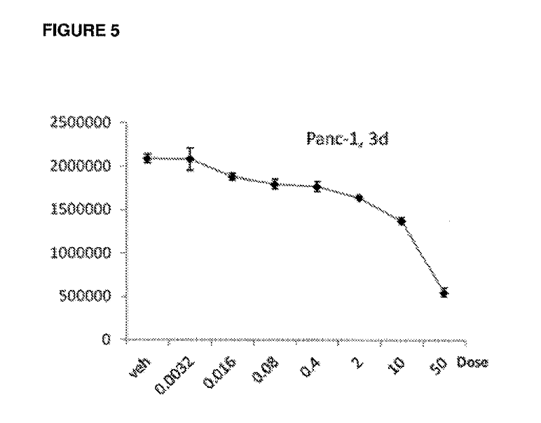

Body weight change curves showing the group median % body weight change as a function of time (days) are presented in FIG. 2.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein is employed in practicing the invention. It is intended that the following claims define the scope of the invention and that methods and structures within the scope of these claims and their equivalents be covered thereby.

1-63. (canceled)

64. A method for (i) treating a proliferative disorder of a plurality of pancreatic cells in an individual; or (ii) treating a pancreatic tumor in a subject with a pancreatic tumor; or (iii) degrading, inhibiting the growth of, inhibiting the proliferation of or killing pancreatic cancer cells; or (iv) slowing the progression of pancreatic carcinogenesis, reversing pancreatic carcinogenesis or inhibiting pancreatic carcinogenesis in a subject; or (v) lowering the risk of developing invasive pancreatic cancer in an individual,
comprising administering to an individual or subject in need thereof a therapeutically effective amount of (S)-N-(3,4-difluoro-2-(2-fluoro-4-iodophenylamino)-6-methoxyphenyl)-1-(2,3-dihydroxypropyl)cyclopropane-1-sulfonamide; or N-(4-(2-fluoro-4-iodophenylamino)-1,5-dimethyl-6-oxo-1,6-dihydropyridin-3-yl)cyclopropanesulfonamide; or a pharmaceutically acceptable salt thereof; or a polymorph thereof.

65. The method of claim 64, wherein in the method for (i) treating a proliferative disorder of a plurality of pancreatic cells in an individual,
the proliferative disorder is a pancreatic cancer, a precancerous condition of the pancreas, hyperplasia of the pancreas, metaplasia of the pancreas, dysplasia of the pancreas, duct-cell carcinoma, pleomorphic giant-cell carcinoma, giant-cell carcinoma (osteoclastoid type), cancer, adenosquamous carcinoma, mucinous (colloid) carcinoma, cystcancer, acinar-cell cancer, papillary cancer, small-cell (oat-cell) carcinoma, pancreaticoblastoma, mixed-cell carcinoma, anaplastic carcinoma, pancreatic hyperplasia, pancreatic metaplasia, pancreatic dysplasia, mucinous cystadenoma, intraductal papillary neoplasm, serous cystadenoma, papillary-cystic neoplasm, mucinous cystic tumor with dysplasia, intraductal papillary mucinous tumor with dysplasia, pseudopapillary solid tumor or metastatic pancreatic cancer, or a combination thereof.

66. The method of claim 64, wherein the administration is parenteral, by injection, intravenous, oral, topical or a combination thereof.

67. The method of claim 64, wherein in the method for (ii) treating a pancreatic tumor,
the tumor is benign or malignant, or
tumor growth rate is reduced, or
an increase in tumor size is prevented, or
tumor size is reduced, or
an increase in tumor volume is prevented, or
the tumor volume is reduced, or
tumor proliferation is prevented, or
tumor proliferation is reduced, or
cell death is induced, or a
apoptosis is induced.

68. The method of claim 64, wherein in the method for (v) lowering the risk of developing invasive pancreatic cancer,
the individual suffers from a disease or condition predisposing the individual to develop an invasive pancreatic cancer, or from diabetes mellitus or pancreatitis, or from a hereditary syndrome, or from hereditary nonpolyposis colorectal cancer (HNPCC) or familial adenomatous polyposis (FAP), or the individual has a gene mutation.

69. The method of claim 68, wherein the individual has a gene mutation in the MSH2, MSH6, MLH1, or APC gene.

70. A method according to claim 64, which is for (i) treating a proliferative disorder of a plurality of pancreatic cells in an individual.

71. A method according to claim 64, which is for (ii) treating a pancreatic tumor in a subject with a pancreatic tumor.

72. A method according to claim 64, which is for (iii) degrading, inhibiting the growth of, inhibiting the proliferation of or killing pancreatic cancer cells.

73. A method according to claim 64, which is for (v) lowering the risk of developing invasive pancreatic cancer in an individual.

74. A method according to claim 64, comprising administering to an individual or subject in need thereof a therapeutically effective amount of (S)-N-(3,4-difluoro-2-(2-fluoro-4-iodophenylamino)-6-methoxyphenyl)-1-(2,3-dihydroxypropyl)cyclopropane-1-sulfonamide; or N-(4-(2-fluoro-4-iodophenylamino)-1,5-dimethyl-6-oxo-1,6-dihydropyridin-3-yl)cyclopropanesulfonamide; or a pharmaceutically acceptable salt thereof.

75. A method for degrading, inhibiting the growth of, inhibiting the proliferation of or killing pancreatic cancer cells comprising contacting the cells with an amount of (S)-N-(3,4-difluoro-2-(2-fluoro-4-iodophenylamino)-6-methoxyphenyl)-1-(2,3-dihydroxypropyl)cyclopropane-1-sulfonamide; or N-(4-(2-fluoro-4-iodophenylamino)-1,5-dimethyl-6-oxo-1,6-dihydropyridin-3-yl)cyclopropanesulfonamide; or a pharmaceutically acceptable salt thereof; or a polymorph thereof.

76. A method according to claim 75, comprising contacting the cells with an amount of (S)-N-(3,4-difluoro-2-(2-fluoro-4-iodophenylamino)-6-methoxyphenyl)-1-(2,3-dihydroxypropyl)cyclopropane-1-sulfonamide; or N-(4-(2-fluoro-4-iodophenylamino)-1,5-dimethyl-6-oxo-1,6-dihydropyridin-3-yl)cyclopropanesulfonamide; or a pharmaceutically acceptable salt thereof.

77. A kit for treating a proliferative disorder of a plurality of pancreatic cells in an individual in need thereof, comprising:
i) (S)-N-(3,4-difluoro-2-(2-fluoro-4-iodophenylamino)-6-methoxyphenyl)-1-(2,3-dihydroxypropyl)cyclopropane-1-sulfonamide; or N-(4-(2-fluoro-4-iodophenylamino)-1,5-dimethyl-6-oxo-1,6-dihydropyridin-3-yl)cyclopropanesulfonamide; or a pharmaceutically acceptable salt thereof; or a polymorph thereof; and
ii) instructions for administration of S)-N-(3,4-difluoro-2-(2-fluoro-4-iodophenylamino)-6-methoxyphenyl)-1-(2,3-dihydroxypropyl)cyclopropane-1-sulfonamide; or N-(4-(2-fluoro-4-iodophenylamino)-1,5-dimethyl-6-oxo-1,6-dihydropyridin-3-yl)cyclopropanesulfonamide; or a pharmaceutically acceptable salt thereof; or a polymorph thereof.

78. A kit according to claim 77, comprising:
i) (S)-N-(3,4-difluoro-2-(2-fluoro-4-iodophenylamino)-6-methoxyphenyl)-1-(2,3-dihydroxypropyl)cyclopropane-1-sulfonamide; or N-(4-(2-fluoro-4-iodophenylamino)-1,5-dimethyl-6-oxo-1,6-dihydropyridin-3-yl)cyclopropanesulfonamide; or a pharmaceutically acceptable salt thereof; and
ii) instructions for administration of S)-N-(3,4-difluoro-2-(2-fluoro-4-iodophenylamino)-6-methoxyphenyl)-1-(2,3-dihydroxypropyl)cyclopropane-1-sulfonamide; or N-(4-(2-fluoro-4-iodophenylamino)-1,5-dimethyl-6-oxo-1,6-dihydropyridin-3-yl)cyclopropanesulfonamide; or a pharmaceutically acceptable salt thereof.
